(19) United States
(12) Patent Application Publication
(10) Pub. No.: US 2003/0087316 A1
(43) Pub. Date: May 8, 2003

(54) Diagnostic markers of liver dysfunction

(76) Inventors: Tony E. Hugli, San Diego, CA (US); Craig M. Jackson, San Diego, CA (US)

Correspondence Address: Lisa A. Haile, J.D., Ph.D., GRAY CARY WARE & FREIDENRICH LLP, 4365 Executive Drive, Suite 1600, San Diego, CA, 92121-2189, US

(21) Appl. No.: 09/922,236

(22) Filed: Aug. 2, 2001

(51) Int. Cl. G01N 33/53; G01N 33/567; C12Q 1/37

(52) U.S. Cl. 435/7.21; 435/23

(57) ABSTRACT

The present invention provides methods and kits for detecting and monitoring liver damage in a subject. The methods and kits rely on the correlation between the presence or increase in a kallikrein-like peptidase in a sample from the subject and liver disease. Additionally, the present invention provides in vivo and in vitro methods for detecting toxicity of a therapeutic agent. Methods for detecting, diagnosing, or monitoring liver damage by measuring a panel of components, including kallikrein-like peptidase, along with other blood enzymes and/or complement components are also provided.

### FIELD OF THE INVENTION

The invention relates generally to methods and kits for detecting liver dysfunction and more specifically to measurement of a peptidase or peptidases synthesized in and secreted from the liver, such as those of the hemostatic and complement systems, as an indicator of liver damage.

### BACKGROUND INFORMATION

Currently, methods for detecting liver disease rely on late stage markers that do not identify liver disease before it is in an advanced stage. This has resulted in a deficiency in modern healthcare related to liver disease management, the importance of which is underscored by the fatal nature of liver disease in many instances. Therefore, there is a need for improved methods for liver screening, monitoring, and diagnosis, especially with respect to early stage disease and damage.

The magnitude of this need is immense and growing as more organ transplants are performed. As of the year 2000, there were at least 4,000 liver transplants per year in the United States. In 1998, there were almost 25,000 deaths caused by chronic liver disease. Furthermore, there are estimated to be millions of individuals experiencing a variety of liver diseases from advancing chronic liver disease to acute toxicity and viral injury. Finally, there are perhaps even more individuals with various stages of liver disease that remain undetected. Consequently, a reliable method for detecting early onset liver damage or monitor progressive liver damage or repair is needed. Such an assay should find wide application in research, pharmaceutical product evaluation, development and clinical medicine.

Results of some studies suggest that complement components may play a useful role in liver disease detection or monitoring. However, it has long been known that complement components C3 and C4 are labile in drawn blood and in stored plasma samples resulting in an elevation of the breakdown factors C3 a and C4a versus time. This in vitro activation of complement is enhanced in certain clinical conditions and is largely responsible for a paucity of reliable in vivo complement activation data in the literature. Recently, it has been reported that blood/plasma samples can be stabilized by adding proteinase inhibitors to prevent the in vitro conversion of these complement factors¹⁻³. However, the enzyme(s) responsible for in vitro conversion of complement proteins, mainly C3 and C4, remains unidentified. Therefore, there remains a need to identify the enzyme(s) responsible for this conversion, and a determination of whether this enzyme may be a useful component of a method for detecting and/or monitoring liver disease.

In previous studies, it was observed that conversion of C3 and C4 in the plasma of some, but not all, orthotopic liver transplant recipients (LTR) was much greater than that in plasma of control (non-transplant) individuals. Therefore, a chromogenic substrate Ac-Ala-Gly-Leu-Thr-Arg-p-nitroanilide (AGLTR-pNA) was designed based on the C4 cleavage site, i.e. the C-terminal portion of the C4a molecule⁽⁵⁾. This substrate was used to measure the level of in vitro peptidase activity and to examine specificity of the peptidase(s) in LTR plasma. However, even with the development of this substrate, the enzyme responsible for cleavage of this substrate in LTR remains unidentified. Furthermore, it is not known whether this enzyme may be a useful component of a method for detecting and/or monitoring liver disease.

It is known that complement is activated during acute and chronic bacterial and viral infections. Elevation in complement activation has been associated with autoimmune diseases such as rheumatoid arthritis, systemic lupus erythematosus sepsis, traumatic injury and multi-organ failure, as well as extracorporeal blood treatments such as cardio-bypass blood oxygenation and renal dialysis. Recent plasma data from organ transplant recipients indicated that extensive complement activation also occurs both during acute rejection episodes and when no clinical signs of rejection are evident. The fact that many LTR plasma exhibited high levels of in vitro complement activation indicated that these plasma represent a source for the complement converting proteinase(s). However, this proteinase(s) remains unidentified.

It has also been shown that the contact system of coagulation is activated immediately after graft revascularization in LTR, but this data only included the first one-hundred minutes after reperfusion of the organ. In a preliminary report, marked peptidase activity was demonstrated in plasma obtained from clinically stable LTR months and even years after the organ had been transplanted. However, the peptidase responsible for this activity as well as the relationship of this peptidase with the complement converting enzyme discussed above remains unidentified.

The present invention utilizes the characterization and identification of the enzyme responsible for at least some of the peptidase activity in LTR, to develop improved methods for detecting and monitoring liver disease. Surprisingly, the peptidase activity described herein, using the C4 analog substrate described above and a number of commercial proteinase substrates, is not the complement converting enzyme.

### SUMMARY OF THE INVENTION

The present invention provides a solution to the longstanding need for improved methods for detecting, monitoring, and diagnosing liver damage resulting from a variety of causative factors. The invention is based on the seminal discovery of unexpectedly high levels of a plasma kallikrein-like peptidase in subjects with liver damage as compared to subjects not having liver damage. Levels of this peptidase are elevated in subjects suffering from at least certain forms of liver disease, including recurrent hepatitis C virus (HCV) infection. Additionally, mannan binding serine protease (MASP) levels, as well as certain other complement components, appear to be elevated as a result of at least some forms of liver disease. Given the initial observation of the elevated kallikrein-like peptidase level in subjects with liver damage, one of skill in the art would be able to detect the level of other components of the hemostatic system, both upstream and downstream from kallikrein-like peptidase, e.g., kallikrein, which would also naturally be elevated upon activation of other components of the coagulation cascade.

The methods and kits of the present invention are useful for monitoring and diagnosing various liver diseases, including early stage tissue injury/organ rejection, certain forms of viral infection, drug toxicity, and alterations in liver function. The methods provide information not currently available in the clinical arena, and are rapid and reproducible, and less expensive than current methodologies. The methods and kits of the present invention detect and monitor tissue injury/pathology at an earlier stage of development than is currently detectable using conventional methods. The methods and kits are especially useful to evaluate therapeutic agents and drugs for their toxicity with respect to liver damage in particular. The early detection of liver disease by the methods of the present invention permits earlier clinical intervention if adverse reactions do occur. Knowledge of early stage organ injury should result in improved treatment modes and reduced overall costs.

In one aspect, the present invention provides a method for detecting liver damage in a subject by contacting a sample from the subject with a kallikrein-like peptidase detection reagent or a detection reagent that identifies a component of the hemostatic and complement systems, e.g., thrombin, that would also be elevated due to activation of the cascade. For purposes of discussion, the invention is described in particular with respect to a kallikrein-like peptidase, e.g., kallikrein, but it is understood that other hemostatic and complement systems components are also included. Interaction of plasma kallikrein-like peptidase in the sample with the plasma kallikrein-like peptidase detection reagent is indicative of liver damage in the subject. In preferred embodiments, the kallikrein-like peptidase is kallikrein. The kallikrein-like peptidase detection reagent may be a substrate cleaved by kallikrein or a kallikrein binding reagent, e.g., an antibody, for example, or an active site-reactive reagent, for example a low molecular weight peptide chloromethylketone to which a moiety suitable for detection of the reacted kallikrein-like enzyme is attached (Bock, PE, Thioester peptide Chloromethyl ketones:reagents for active site-selective labeling of serine proteinases with spectroscopic probes, Methods Enzymol, 222:478, 1993).

In certain preferred embodiments, the sample is also contacted with an alpha-2-macroglobulin detection reagent. The assay may detect, for example, kallikrein bound to alpha-2-macroglobulin; free kallikrein/prekallikrein; both bound and free kallikrein; or only prekallikrein. Further, the methods of the invention may include further contacting a sample with a C1 inhibitor detection reagent, an alpha 1 antitrypsin detection reagent, an antithrombin detection reagent, or any combination thereof.

For certain embodiments wherein the kallikrein-like peptidase detection reagent is a substrate and the kallikrein-like peptidase is kallikrein, the kallikrein-like peptidase detection reagent comprises a peptide with a tri, tetra or penta peptide amino acid sequence, e.g. YZR, XYZR, WXYZR, for example, AGLTR, bound to a detectable moiety. Preferably, the P1, or C-terminal position of the peptide sequence is an Arg residue (R). Peptides useful as peptidase detection reagents include those as described in U.S. Pat. No. 4,016,042, herein incorporated by reference. For example, peptides include a synthetic substrate having the structure R1-Pro-X—Y—NH—R2, wherein R1 is a blocking group, —NH—R2 is a chromogenic or fluorescent group, X represents a phenylalanyl, beta-cyclohexylanyl, phenylglycyl or tyrosyl group, and Y represents a protonized arginyl or lysyl group, is disclosed. This substrate is useful for the quantitative determination of proteolytic enzymes of class E.C. 3.4.4., except thrombin and thrombin-like enzymes. Also included are commercially available substrates that are relatively kallikrein specific, such as H-D-But-Cyclohexylalanyl-Arg-pNA or H-D-Pro-Phe-Arg-pNA, for example. pNA could be replaced by a fluorophore, or a moiety detectable by an antibody in an immunoassay, for example.

In certain preferred embodiments, the method further involves contacting the sample with a C4a detection reagent, wherein elevated levels of C4a is indicative of autoimmune liver damage.

In another aspect, the present invention provides a kit having a first container containing a kallikrein-like peptidase detection reagent. The kit may optionally have a second container containing an alpha-2-macroglobulin detection reagent, and/or a second container containing the C4a detection reagent for example.

In another aspect, the invention provides a method for monitoring the progression of liver damage in a subject. The method compares changes in kallikrein-like peptidase levels in a sample over time, using a kallikrein-like peptidase detection reagent over time intervals. In this monitoring aspect of the invention, a change in the relative quantity of the kallikrein-like peptidase is indicative of a change in liver damage state.

In another aspect, the present invention provides a method for detecting kallikrein in a sample by contacting the sample with an isolated peptide having an amino acid sequence consisting essentially of, or consisting of, the sequence AGLTR, with a detectable, e.g., chromogenic, moiety bound at the arginine residue. In certain preferred embodiments, the chromogenic moiety is pNA.

In another aspect, the present invention provides a method for determining toxicity of an agent, e.g., a drug, in a subject utilizing a kallikrein-like peptidase detection reagent. Kallikrein-like peptidase levels in the sample are determined using the kallikrein-like peptidase detection reagent. An elevated kallikrein-like peptidase level as compared to a control, non-treated sample, indicates toxicity of the agent.

In another aspect, the present invention provides a panel of assays including a kallikrein-like peptidase detection assay, for use in detecting, diagnosing, and/or monitoring liver disease. In preferred embodiments, the kallikrein-like peptidase detection assay is a kallikrein detection assay. The assay panel provides a greater understanding of the types and stages of disease, along with an improved ability to detect, diagnose, and monitor liver disease, as discussed above for other aspects of the invention. In certain preferred embodiments, the panel assay combines the data for complement activation as a window on various types of immune injury and kallikrein and/or prekallikrein measurements as a window of injury produced by certain types of viral infections. In preferred embodiments, the measurements of the panel are performed at one time, most preferably using an automated instrument.

In general, this aspect of the invention provides a method for detecting liver damage in a subject by contacting a sample from the subject with a series of detection reagents that are specific for each member of a liver damage panel that includes a reagent for kallikrein-like peptidase. Preferably, the kallikrein-like peptidase is kallikrein. Furthermore, the panel typically includes detection reagents for one or more complement components. An elevated level of one or more members of the liver damage panel is indicative of liver damage in the subject.

In another aspect, the present invention provides an in vitro method for screening a therapeutic agent for toxicity. The method involves determining the level of kallikrein-like peptidase, preferably kallikrein, in an in vitro assay after incubating cultured cells, e.g., hepatocytes, in the presence of the therapeutic agent. The presence of kallikrein-like peptidase levels, are determined using a kallikrein-like peptidase detection reagent. Preferred embodiments of this aspect of the invention are described above for the section describing methods of the present invention for detecting liver damage.

### BRIEF DESCRIPTION OF THE FIGURES

FIG. 1 is a graph of AGLTR-pNA hydrolyzing activities determined in EDTA plasma samples from 16 LTR and 16 non-transplant donors (NTD). The EDTA plasma samples were diluted 10-fold with TBS-EDTA and activity was determined by reading at 405 nm after incubation at 37° C. for 15 min. Each point is the average of values determined in duplicate wells. LTR and NTD proteinase levels differed at a statistical significance of P<0.01 using the Mann-Whitney U-test.

FIG. 2 is a graph of AGLTR-pNA hydrolyzing activity measured in LTR (panel A) and in NTD (panel B) plasma samples versus time at 37° C. The LTR (n=16) samples were diluted 10-fold in TBS-EDTA and the NTD (n=14) samples were undiluted. Each point represents an average value from duplicate determinations at the specified time points. Trypsin (16 nM) was used as a positive control.

FIG. 3 is a plot of the AGLTR-pNA hydrolyzing activity in the LTR samples after 15 min at 37° C. versus the C4a concentrations (i.e. C4 converting activity) in EDTA plasma samples from the same LTR samples (n=15). Each value represents an average of duplicate measurements. No correlation was observed between the peptidase activity and the C4a levels in these LTR plasmas.

FIG. 4 is an absorbance profile of Gel filtration samples of a LTR plasma separated on a Sephacryl S-300 column. The fractions from the column were incubated with substrates at 37° C. for 15, 35 and 45 min prior to measuring the OD at 405 nm. Elution profiles for the protein (OD 280 nm) and peptidase activity is shown. Substrates AGLTR-pNA, S2288, and S2302 were used.

FIG. 5 is an absorbance profile of Gel filtration samples of NTD plasma separated on a Sephacryl S-300 column. The fractions were incubated with substrates at 37° C. for 15, 80 and 180 min prior to measurement of OD at 405 nm. Elution profiles for the protein (OD 280 nm) and peptidase activity are shown. Substrates AGLTR-pNA and S2288 were used.

FIG. 6 is a graph of an immuno-dot blot and double-immunodiffusion analysis of the gel filtration fractions of LTR plasma. Plasma kallikrein was detected in each fraction by immuno-dot blot and expressed as relative intensity (-0-). The α2M, C1INH and AT III were detected in each fraction by double-immunodiffusion. The thickness of the bar indicates the relative intensity of the immunoreaction (from ±, thin line to +++, thick line). The peptidase activity was detected using the substrate AGLTR-pNA and is indicated by solid circles (-●-).

FIG. 7 is a series of bar graphs of protease activity of various proteinases at 10 nM incubated with three different substrates at 37° C. for 15 min. Plasma kallikrein and trypsin cleaved AGLTR-pNA relatively efficiently, but neither C1s nor any of the other proteinases efficiently cleaved this substrate. All of the substrates were hydrolysed by plasma kallikrein and trypsin, while thrombin preferred S2288 over either AGLTR-pNA or S2302.

FIG. 8 is a series of bar graphs of proteinase activity where the thrombin inhibitor hirudin was used to attempt to inhibit the proteinase in LTR plasma. The plasma was incubated with 50 U/ml hirudin and assayed using the substrate AGLTR-pNA versus time at 37° C. Although thrombin was effectively inhibited by hirudin, the peptidase activity in LTR plasma was not.

FIG. 9 is a photograph of an immunoblot of plasma kallikrein and plasma kallikrein-inhibitor complexes in plasma. Lane 1, plasma kallikrein (100 nM); lane 2, plasma kallikrein (100 nM) was incubated with C1INH (200 nM) at RT for 30 min; lane 3, plasma kallikrein (100 nM) was incubated with alpha-2-macroglobulin (500 nM) at RT for 30 min; lane 4, NTD plasma; lane 5, LTR plasma. The numbers to the left of the gel represent mol wt standards in kD.

FIG. 10 is a photograph of SDS PAGE analysis of cleavage of C4 by various purified enzymes. A constant concentration of C4 (1 μM) was incubated with 40 nM of each enzyme for 60 min at 37° C. The C4 was analyzed by 10% SDS-PAGE under reducing conditions. Lane 1, markers; lane 2, no enzyme (C4 only); lane 3, C1s with 10 mM EDTA; lane 4, C1s with 10 mM CaCl₂; lane 5, C1s with 10 mM CaCl₂ and 10 mM MgCl₂; lane 6, plasma kallikrein with 10 mM EDTA; lane 7, plasma kallikrein with 10 mM CaCl₂; lane 8, plasma kallikrein with 10 mM CaCl₂ and 10 mM MgCl₂.

FIG. 11 is a graph illustrating the relationship between recurrence of HCV and the peptidase activities in EDTA plasmas from LTR. LTR were divided into the recurrence positive and negative groups. The values for the Peptidase activity shown in FIG. 1 were used. The Mann-Whitney U-test was used for statistical analysis.

### DETAILED DESCRIPTION OF THE INVENTION

The present invention provides methods for detecting liver disease in a sample from a subject. The methods rely on the correlation between the presence or increase in a kallikrein-like peptidase and liver disease. The methods detect or measure kallikrein-like peptidase by detecting interactions between kallikrein-like peptidase and a kallikrein-like peptidase detection reagent. It should be understood that the invention methods are also useful for detection of components that are synthesized in the liver or derived by activation processes from proteins synthesized in the liver. Such components include, from coagulation, thrombin(prothrombin), factor Xa(factor X), factor IXa(factor IX), factor XIa(factor XI), factor XIIa(factor XII), factor VIIa(factor VII), activated protein C(protein C) as well as kallikrein(prekallikrein); from fibrinolysis, plasmin(plasminogen). In a particular illustrative example, the invention methods utilize kallikrein for detection of liver damage.

The kallikrein-like peptidase detected by the method of the present invention is identified by its peptidase activity or presence in the plasma of certain liver transplant patients, especially those with recurrent viral infection, as illustrated in the Examples section below. In a particular Example provided herein, the peptidase activity of the kallikrein-like peptidase cleaved a synthetic substrate, AGLTR-pNA, to release a chromogenic pNA moiety. Additionally, the peptidase activity is present in molecular complexes of about 100-2000 kDa. The activity of the kallikrein-like peptidase detected by the present invention is substantially inhibited by PPACK II, inhibited partially by CINH, but not inhibited by hirudin. Unless otherwise indicated, the term “kallikrein-like peptidase” in the present specification refers to the peptidase with the activity and characteristics described above.

As exemplified in the Examples section, one coagulation cascade component, a peptidase detected by the kits and methods of the present invention, is a kallikrein-like peptidase, preferably kallikrein. Plasma kallikrein is a serine protease involved in blood coagulation and a variety of other processes as well. Kallikrein cleaves high molecular weight kininogen (HMWK) to form bradykinin, a potent vasodilator and endothelial cell activator.

Kallikrein is found in plasma in a precursor form as well as a complexed form. Prekallikrein, the precursor of kallikrein, is a glycoprotein comprised of a single polypeptide chain with a molecular weight of 80,000 Da. Kallikrein consists of 2 disulfide bonded chains of 43,000 and 33,000-36,000 Da. The light chain of kallikrein contains the enzymatic domain while the heavy chain appears to be required for surface dependent activation of coagulation. The sequence of human plasma kallikrein is known (See NCBI Accession P03951, herein incorporated by reference).

Kallikrein binds to a number of proteins. In blood, most prekallikrein circulates bound to high molecular weight kininogen (HMWK). Additionally, kallikrein complexes with other proteins, for example, with C1INH to form kallikrein-C1INH complexes, and with alpha-2-macroglobulin to form kallikrein-alpha-2-macroglobulin complexes as described in further detail below.

The methods of the present invention may be used to analyze liver damage in a subject. Typically, the subject is an animal, in certain preferred embodiments, a mammal, for example, a human. The assay is particularly effective in situations where the subject is at risk for developing liver disease, for example, where the subject is infected with a hepatitis virus.

Liver disease is any disease, disorder, or damage that causes the liver or liver cells, e.g., hepatocytes or endothelial cells, to function improperly or cease functioning. Traditionally, late stage liver disease is detected by abnormal liver function tests. Liver diseases include, but are not limited to the following: liver abscess, liver cancer, either primary or metastatic, cirrhosis, such as cirrhosis caused by the alcohol consumption or primary biliary cirrhosis, amebic liver abscess, autoimmune hepatitis, biliary atresia, coccidioidomycosis disseminated, delta agent (hepatitis d), hemochromatosis, hepatitis a, hepatitis b, hepatitis c, or other hepatitis virus, hepatocellular carcinoma, pyogenic liver abscess, Reye's syndrome, sclerosing cholangitis, Wilson's disease, drug induced hepatotoxicity, and fulminant or acute liver failure. In certain preferred embodiments, the liver damage detected by the methods of the present invention is the result, at least in part, of recurrent HCV infection.

The assays of the present invention utilize a kallikrein-like peptidase detection reagent. In preferred embodiments, the kallikrein-like peptidase detection reagent is a kallikrein detection reagent. The kallikrein detection reagent can be virtually any reagent useful for detecting a protein and/or peptidase enzyme activity, many of which are known in the art. Typically, the kallikrein detection reagent is a substrate for kallikrein enzymatic activity or a kallikrein-binding reagent, such as an anti-kallikrein antibody.

In embodiments where the detection agent is an antibody, methods of detection include immunoassays, which are well known in the art. Virtually any format of immunoassay can be used with the invention methods wherein the kallikrein-like peptidase is at least one of the components detected. Preferably, the immunoassay is performed on an automated analyzer such as, but not limited to, a Luminex 100 (Luminex, Austin, Tex.).

Antibodies, especially monoclonal antibodies, that specifically recognize kallikrein in both a free and complexed form (e.g. 13G11, available from QED Biosciences Inc., San Diego, Calif.) (27 and 28), or recognize only prekallikrein (Enzyme Research Labs, Inc., South Bend, Ind.) are available commercially. Methods for generating antibodies specific for free kallikrein, prekallikrein, or complexed kallikrein are known in the art or can be generated by standard methods. Monoclonal antibodies are made from antigen containing fragments of the protein by methods well known to those skilled in the art (Kohler, et al., Nature, 256:495, 1975). The term antibody as used in this invention is meant to include intact molecules as well as fragments thereof, such as Fab and F(ab′)₂, Fv and SCA fragments which are capable of binding an epitopic determinant on kallikrein or other coagulation cascade component to be detected.

In embodiments in which the kallikrein-like peptidase detection reagent is a substrate, interaction of the substrate with the kallikrein-like peptidase is detected by detecting cleavage of the substrate. For example, the substrate may include a chromogenic moiety attached to a peptide substrate by a bond that is cleaved by the kallikrein-like peptidase.

In one EXAMPLE provided herein, a substrate that is an isolated peptide consisting essentially of the sequence AGLTR, most preferably consisting of the sequence AGLTR, bound at the arginine residue to a chromogenic moiety is utilized. For certain embodiments wherein the kallikrein-like peptidase detection reagent is a substrate and the kallikrein-like peptidase is kallikrein or other peptidase, the kallikrein-like peptidase detection reagent comprises a peptide with a tri, tetra or penta peptide amino acid sequence, e.g. YZR, XYZR, WXYZR, for example, AGLTR, bound to a detectable moiety. Preferably, the PI, or C-terminal position of the peptide sequence is an Arg residue (R). Peptides useful as peptidase detection reagents include those as described in U.S. Pat. No. 4,016,042, herein incorporated by reference. For example, peptides include a synthetic substrate having the structure R1-Pro—X—Y—NH—R2, wherein R1 is a blocking group, —NH—R2 is a chromogenic or fluorescent group, X represents a phenylalanyl, beta-cyclohexylanyl, phenylglycyl or tyrosyl group, and Y represents a protonized arginyl or lysyl group, is disclosed. This substrate is useful for the quantitative determination of proteolytic enzymes of class E.C. 3.4.4., except thrombin and thrombin-like enzymes. Also included are commercially available substrates that are relatively kallikrein specific, such as H-D-But-Cyclohexylalanyl-Arg-pNA or H-D-Pro-Phe-Arg-pNA, for example. pNA could be replaced by a fluorophore, or a moiety detectable by an antibody in an immunoassay, for example.

“Consisting essentially of the sequence” means that the peptide may have a sequence that includes any tri, tetra or penta peptide for example, such as, AGLTR, and/or may include additional amino acid residues, provided that the sequence is not the entire sequence of complement component C4, and provided that the sequence retains the ability to be cleaved by kallikrein or other peptidase as described herein. The term “isolated” peptide refers to is intended a peptide molecule which is free of other proteins, lipids, nucleic acid and other molecules and has been removed from its native environment. For example, preferred isolated peptides used as substrates in the methods of the present invention are synthetically produced, as is known in the art. A number of available FMOC peptide synthesis systems are available. For example, assembly of a polypeptide or fragment can be carried out on a solid support using an Applied Biosystems, Inc., Model 431A automated peptide synthesizer. Such equipment provides ready access to the peptides of the invention, either by direct synthesis or by synthesis of a series of fragments that can be coupled using other known techniques. Accordingly, methods for the chemical synthesis of polypeptides and peptides are well-known to those of ordinary skill in the art, e.g., peptides can be synthesized by solid phase techniques, cleaved from the resin and purified by preparative high performance liquid chromatography (see, e.g., Creighton, 1983, Proteins: Structures and Molecular Principles, W. H. Freeman & Co., N.Y., pp. 50-60). The composition of the synthetic peptides can be confirmed by amino acid analysis or sequencing; e.g., using the Edman degradation procedure (see e.g., Creighton, 1983, supra at pp. 34-49). Thus, fragments of the cleavage site, functional variants, or mutants can be chemically synthesized.

Many detectable labels, reporters, moieties are known in the art and can be used with the invention peptides provided that they can be attached to the peptide with a bond that is cleavable by a peptidase such as kallikrein. For example, the detectable moiety may be chromogenic, fluorogenic, or luminescent, or may be a member of a specific binding pair, a substance detectable by an antibody in any of the known immunoassay methods. There are many different labels and methods of labeling known to those of ordinary skill in the art. Examples of the types of labels which can be used in the present invention include enzymes, radioisotopes, fluorescent compounds, colloidal metals, chemiluminescent compounds, phosphorescent compounds, and bioluminescent compounds. Those of ordinary skill in the art will know of other suitable labels for binding to the peptide or to an antibody, or will be able to ascertain such, using routine experimentation. In one embodiment, the chromogenic moiety is p-nitroanilide (referred to herein as pNA).

In certain embodiments, the kallikrein-like peptidase level is determined, or the presence or absence of the kallikrein-like peptidase is determined, by comparing kallikrein-like peptidase level of the sample with a kallikrein-like peptidase level of a control containing no kallikrein-like peptidase. In other embodiments, the kallikrein-like peptidase level is determined by comparing the amount of kallikrein-like peptidase bound to a kallikrein-like protease detection reagent with that of one or more of a series of standards containing known amounts of kallikrein or kallikrein-like protease. Assay strategies using controls and/or standards are well known in the art.

Typically, samples analyzed using the methods of the present invention are blood-based samples. Preferably, the blood-based samples are plasma samples. Methods for obtaining plasma samples from blood samples are well known in the art. For example, plasma may be isolated from whole blood samples using EDTA tubes (Venoject; Terumo Corp., Elkton, Md.) as illustrated in the Examples section. The plasma samples may be either processed immediately for analysis or stored at −70° C. Frozen samples are typically thawed at 4° C. prior to analysis.

As indicated in the Examples provided hereinafter, in certain preferred embodiments of the present invention, kallikrein is detected bound with alpha-2-macroglobulin. In certain preferred embodiments, methods of the present invention detect kallikrein associated with alpha-2-macroglobulin separately from prekallikrein or other forms of kallikrein that are not associated with alpha-2-macroglobulin. Such assays may be designed using methods that are well known in the art. For example, while not intended to be limiting, antibodies that specifically recognize alpha-2-macroglobulin are known in the art and available commercially (e.g. Enzyme Research Labs, Inc. South Bend, Ind.). Alpha-2-macroglobulin is well-known in the art. Other inhibitors that may be bound to kallikrein, for example, and detected include alpha-2-macroglobulin, C1 or alpha-1 antitrypsin inhibitor.

In certain embodiments, the methods of the present invention include one or more kallikrein-like peptidase inhibitor. These methods are expected to increase the accuracy of the methods of the present invention. In methods in which inhibitors are used, the sample is typically divided into a first portion and a second portion before contacting a sample with a substrate. A kallikrein-like peptidase inhibitor, preferably a kallikrein peptidase inhibitor, is then added to the first portion of the sample before contacting the sample with the substrate. Confirmation that the measured peptidase activity is the activity of kallikrein-like peptidase identified in the present specification, is provided by a diminished level of kallikrein-like peptidase activity in the first portion, which contains the kallikrein-like peptidase inhibitor. This inhibitor preferentially inhibits kallikrein-like peptidase over other blood peptidases, and preferably is specific for kallikrein-like peptidase, and/or in preferred embodiments, kallikrein peptidase activity. In certain preferred embodiments, the kallikrein peptidase inhibitor is SBTI, aprotinin, or preferably PPACK II.

In another aspect, the present invention is useful for monitoring liver dysfunction or disease. The methods are similar to those described above for detecting liver disease except that the test is performed repeatedly over time intervals. Values obtained for the kallikrein-like peptidase or kallikrein peptidase may be compared between timepoints to assess the status of liver disease in a subject. Alternatively, the value at each timepoint may be analyzed independently related to a threshold level indicating the presence of liver disease. More specifically in these embodiments the absolute or relative quantity of kallikrein-like peptidase in a first sample and a second sample are compared by determining interaction of the kallikrein-like peptidase with the kallikrein-like peptidase detection reagent in the samples. A change in the absolute or relative quantity of the kallikrein-like peptidase is indicative of a change in liver disease state.

In preferred embodiments, the present invention provides a method for determining toxicity of a therapeutic agent or drug in a subject by contacting a sample from the subject with a kallikrein-like peptidase detection reagent and determining a kallikrein-like peptidase level in the sample. The presence or relative quantity of kallikrein-like peptidase provides information regarding toxicity of the therapeutic agent.

Although toxicity testing using the methods of the present invention is useful in assessing the toxicity of an agent to a subject, the methods are particularly useful in improving toxicity testing of new or previously untested therapeutic agents, for example, during clinical trials. In certain preferred embodiments, the methods are used initially to test toxicity of a therapeutic agent in animals, especially those of a mammalian species other than humans. If acceptable, non-toxic reports are generated in animals, the methods are then typically used in early phase human clinical trials. Thus, the methods of the present invention provide an earlier and more sensitive detection of toxicity of a therapeutic agent.

Preferred embodiments of the toxicity test methods of the present invention differentiate liver damage due to toxicity from liver damage having an immunological etiology. The invention methods may detect liver damage by detecting and/or measuring kallikrein-like peptidase using the methods of the present invention described above, in combination with an assay that measures levels of certain complement components, particularly C3a and C4a in combination. Methods for determining complement component levels are known in the art. The Examples section of the present specification provides non-limiting examples of a method for measuring C3a levels (further, see U.S. Pat. No. 6,235,494, herein incorporated by reference). Preferably, methods are used that simultaneously measure the kallikrein-like peptidase and one or more complement components, especially C3a and C4a. The panel testing section below, describes methods, as are known in the art, for simultaneously measuring the levels of several blood analytes.

In one embodiment, elevated levels of kallikrein-like peptidase indicate liver damage due to toxicity, whereas elevated levels of complement components indicate an immunological basis of liver disease. These embodiments will help to assure that toxic agents are identified, while agents that are being evaluated while a patient develops an unrelated immunologically-induced liver disease are not necessarily identified as toxic. Therefore, the present methods provide tremendous benefits to the development of safe therapeutic agents.

Embodiments for toxicity testing utilizing levels of the kallikrein-like peptidase identified in the present specification, are similar to those described above. The methods for toxicity testing are similar to those described above except that samples analyzed typically include at least one, and preferably a series of samples taken from a subject at different time points after a therapeutic agent has been administered to the subject. Toxicity testing by methods of the present invention utilizes the monitoring methods of the present invention. In certain preferred embodiments, the therapeutic agents are pharmaceutical compounds.

In another aspect, the present invention provides kits for carrying out the methods of the present invention. Optionally, the kit includes an instruction manual for carrying out the methods and a carrier for holding and transporting a first container containing a kallikrein-like peptidase detection reagent, such as a peptide as described above, or an antibody that binds to kallikrein-like peptidase. In certain preferred embodiments the kallikrein-like peptidase is plasma kallikrein. The kit may include a second container containing another reagent that is used for certain preferred embodiments of the methods of the present invention. For example, the kit may include a second container with an alpha-2-macroglobulin detection reagent, a C4a detection reagent, a control comprising kallikrein-like peptidase, a kallikrein/alpha-2-macroglobulin complex control, an active site reactive reagent such as a peptide chloromethylketone, or a kallikrein-like peptidase control in a form effective for interacting or competing with the kallikrein-like peptidase for a detection or binding reagent. Preferably when C3a or C4a detection reagents are utilized, they are used together in combination to provide a more discriminating result.

In another aspect, the present invention provides in vitro methods for determining toxicity of a drug or therapeutic agent. For methods of this aspect of the invention, liver cells, e.g., hepatocytes or endothelial cells, are incubated in cell culture medium under conditions in the presence of the drug or agent for a time sufficient to allow the drug or agent to interact with the cells. Effective culture conditions include, but are not limited to, effective media, temperature, pH, and oxygen conditions wherein liver cells secrete kallikrein-like peptidase in the presence of a drug or therapeutic agent that causes liver damage or liver dysfunction.

Liver cells for this aspect of the invention include any liver cell which produces kallikrein-like peptidase or kallikrein when exposed to drugs or therapeutic agents that may exhibit a toxic effect on such cells. Methods for culturing primary liver cells and liver cell lines are known in the art. Mammalian liver cell lines are commercially available (American Type Culture Collection, Manassus, Va.).

The cell culture medium utilized for this aspect of the invention is an effective medium. An effective medium refers to any medium in which a liver cell performs fundamental processes and proliferates if the cell is not fully differentiated. Such medium typically includes an aqueous medium having carbon, nitrogen and phosphate sources, and appropriate salts, minerals, metals and other nutrients, such as vitamins. Additionally, such medium typically includes serum or a source of required serum factors. Where serum is used in the tissue culture medium, such serum should preferably be prekallikrein or plasma kallikrein-deficient or free so as not to adversely affect results of the method. One of skill in the art would understand that the culture media utilized for in vitro methods of the invention should be substantially free of other interfering proteinases or proteinase precursors or treated to remove or inactivate proteinase or proteinase precursors deleterious to the performance of the method of the invention.

Commercial sources of liver cell lines typically include information regarding suggested cell culturing conditions and media.

Cells of the present invention can be cultured in virtually any standard tissue culture container, especially microtiter dishes for screening many on-test therapeutic agents in the same experiment. Culturing can be carried out at a temperature, pH, and oxygen conditions appropriate for a recombinant cell. Such culturing conditions are within the expertise of one of ordinary skill in the art. Preferred host cells are described above.

After culturing the liver cells in the presence of the toxic compound the sample cell culture medium is collected using standard culture medium collection techniques, thereby separating the sample cell culture medium from the cells. This separation may be carried out utilizing the adhesion of cells to tissue culture containers onto which the cells have adhered during culture, or by utilizing gravity or centrifugation to pellet the cultured liver cells, as is known in the art. The phrase “collecting the sample cell culture medium”, as well as similar phrases, refers to collecting the whole medium which may contain kallikrein-like peptidase. Kallikrein-like peptidase typically need not be further enriched or purified for the in vitro methods of the present invention.

The final steps for in vitro toxicity testing methods of the present invention involve contacting the sample cell culture medium with a kallikrein-like peptidase detection reagent, and detecting interaction of kallikrein-like peptidase in the sample cell culture medium with the kallikrein-like peptidase detection reagent. Elevated levels of interaction of the kallikrein-like peptidase with the kallikrein-like peptidase detection reagent is indicative of therapeutic agent toxicity. These final steps for in vitro test methods of the present invention involving contacting and detecting an interaction are identical to the contacting and detecting steps for other methods of the present invention except that the sample is derived from cell culture medium instead of blood.

In preferred embodiments of the in vitro test methods, as discussed above for other aspects of the invention, the kallikrein-like peptidase is kallikrein. As described above in relation to other aspects of the invention, the kallikrein-like peptidase detection reagent may be a substrate cleaved by kallikrein-like peptidase, or kallikrein, or it may be a kallikrein binding reagent such as an anti-kallikrein antibody.

In addition, preferred embodiments described above in relation to the methods for detecting liver damage, may preferentially be included in the in vitro test methods as well. For example, the method can include steps for detecting alpha-2-macroglobulin and/or kallikrein bound to alpha-2-macroglobulin.

In certain preferred embodiments, in vitro methods of the present invention utilize control cell culture medium prepared in an identical manner to sample cell culture medium except that control cells are not exposed to the therapeutic agent. kallikrein peptidase levels in control cell culture medium is then compared to levels in sample control culture medium. Increased levels of of the kallikrein-like peptidase in the sample culture medium is indicative of therapeutic agent toxicity.

In certain preferred embodiments, the methods and kits of the present invention detect, monitor, or diagnose liver damage by detecting and/or quantitating kallikrein-like peptidase along with various complement components and/or blood enzymes as part of a liver damage panel test. In certain preferred embodiments, the liver damage panel test combines the data for complement activation to detect immune injury and kallikrein and/or prekallikrein measurements to detect injury produced by certain prominent types of viral infections, or other types of liver damage other than immunological damage. In preferred embodiments, the liver damage panel of assays are performed at one time, most preferably using an automated instrument.

In general, this aspect of the invention provides a method for detecting liver damage in a subject by contacting a sample from the subject with a series of detection reagents that are specific for each member of a liver damage panel that includes a kallikrein-like peptidase. Preferably, the kallikrein-like peptidase portion of this assay measures plasma kallikrein. Furthermore, the panel typically includes one or more complement component, such as C3a, C4a and particularly in combination. An elevated level of one or more members of the liver damage panel is indicative of liver damage in the subject.

The liver damage panel consists of selected plasma proteins. The panel testing aspect of the invention includes testing, preferably simultaneous testing, for kallikrein and/or prekallikrein, and at least one other liver damage panel member, preferably a complement component. In a most preferred embodiment of this aspect of the invention, the panel testing includes an assay for each of the members of the liver damage panel.

In one illustrative example, the liver damage panel members include complement components C3a, C4a, C1s and MASP-1, plasma prekallikrein and kallikrein/inhibitor (e.g. alpha-2-macroglobulin and C1 inhibitor) complexes, as well as serum amyloid protein (SAP) and/or C-reactive protein (CRP) representing acute phase proteins. Commercially available monoclonal or polyclonal antibodies exist to all of these components (R&D Systems, Minneapolis, Minn.; Affinity Biologicals; Quidel, San Diego, Calif.; Advanced Research Technologies, San Diego, Calif.; Pharmingen, San Diego, Calif.).

As mentioned above, the panel testing aspect of the present invention not only can be used to detect and monitor liver disease, it provides the ability to discriminate amongst various forms of liver disease and various types of complement activation. The blood complement system can be activated through three independent processes, namely the classical, alternative, and lectin pathways³⁹. The classical pathway results in the generation of both fragments C3a and C4a when the enzyme C1s is activated by interaction of C1 with altered IgG molecules as in immune complexes, this process reflects immune injury. The alternative pathway results in only C3a generation (not C4a) and is activated mainly by invasion of pathogens such as bacteria, viruses, fungi and parasites. The lectin pathway⁴⁰ is activated by the binding of a mannose binding protein (MBP) to exposed terminal neutral sugars such as mannose and generates both C3a and C4a. However, the enzyme MASP-1 must also be activated. The mannose is generally exposed by the action of sialidases such as neuraminidase on either host or invading cells, and this process can occur during an early stage injury event in the vasculature. Therefore, measurement of C3a, C4a, C1s and MASP-1 not only indicate the level or extent of complement activation; these results also identify the pathway or pathways that have been activated. This quality of data can provide insight into the nature of the injury process⁴³⁻⁴⁴. These assays can therefore preferentially discriminate among the three activation pathways of complement.

Plasma protein complexes formed by activated kallikrein and alpha-2-macroglobulin provide evidence of “contact activation”, processes similar to those commonly associated with the coagulation cascade. As demonstrated in the attached Examples section, the presence and quantity of α2-macroglobulin-kallikrein complexes correlate significantly with hepatitis C (HCV), and possibly HBV, infection⁴¹. Not to be limited by theory, in the case of HCV viremia, it is probable that direct injury of the hepatocytes leads to the conversion of prekallikrein to kallikrein. It is well known that HCV infects hepatocytes and causes injury to this cell type⁴². Consequently, we hypothesize that other forms of liver injury, particularly those causing hepatocyte injury, will activate prekallikrein and result in the formation of alpha-2 macroglobulin-kallikrein complexes.

Measurement of acute phase proteins, such as serum amyloid protein and/or C-reactive protein provide independent indicators of the acute phase response and a measure of the extent of upregulation of the host response to endotoxins or other systemic stimuli. Such additional markers provide further data capable of discriminating among the types of agents or mechanisms causing liver injury.

The panel testing methods of the present invention can be used to detect the onset of liver injury, to provide evidence of liver injury or toxicity, to monitor the efficacy of general treatment modes, and presumably also indicate resolution of an existing injury or pathologic condition. These markers are more sensitive than are the classic liver enzyme markers that rely on gross functional alterations of the organ.

Complement activation reflects not only the process of organ rejection but should detect subtle and early stage tissue injury caused for example by therapeutic drugs. Since it is known that immune injury can be detected by the classical pathway and inflammatory injury is signaled by the alternative pathway, these conditions are monitored using the proposed panels. The lectin pathway may be able to detect host tissue injury, such as that caused by toxic drug effects. This information would help diagnose drug side effects, particularly in the liver. A major advantage of this approach using blood samples to detect and monitor the status of the liver, is avoiding the need for needle biopsies.

Until recently, it had not been shown that plasma samples from patients with high levels of in vivo complement activation could be drawn and stabilized for later analysis, without encountering extensive in vitro activation that could invalidate the results. Thus measurements of complement components and/or activation products are seldom used as a diagnostic tool today. This lack of utility of complement assays is due to the fact that enzymes, even in EDTA plasma from “normal” individuals, continue to generate complement fragments in vitro. This process has invalidated many results from previous clinical complement studies. The in vitro activation process can be much greater in patient plasma than in plasma from a healthy donor.

A recent study by Hugli et al.¹ has demonstrated that this problem can be overcome by the use of defined levels of the proteinase inhibitor nafamostat mesilate, commonly known as Futhan, in combination with EDTA. This combination of inhibitors can prevent the in vitro activation of complement by any of the three known pathways. Plasma samples that are properly collected and stored can provide accurate measurements of in vivo levels of complement fragments free of any contribution from in vitro activation. This procedure of sample handling using the above-identified inhibitors is critical for any embodiments of the present invention involving the measurement of complement components. Preferably, the sample handling technique described above by which several inhibitors are added to a sample, is used for all of the methods of the present invention. This sample-handling process permits baseline levels of the complement activation factors to be determined in plasma from normal (i.e. the controls) healthy individuals. When samples are collected properly, the elevation in C3a and C4a levels in the patient samples are many times higher than background or normal levels, thus avoiding false positive data.

The panel aspect of the invention has many important uses. For example the panel is useful for evaluating the effects of drugs under development in producing liver injury and toxicity. The panel of assays is useful in evaluating efficacy of drug treatments such as anti-virals (HCV and HBV) to treat hepatitis and anti-rejection regimens in liver transplant patients. The panels may be used as a pre-clinical monitoring tool for use in validating clinical trials or as a clinical diagnosis tool for detecting liver injury and liver disease. Finally, the panel of assays may be used as a clinical monitoring tool for following the progression or resolution of liver injury and disease including side effects from drug treatments.

In certain preferred embodiments of the panel testing aspect of the present invention, the testing is performed using a high-throughput system. For Example, the Luminex bead technology system (Luminex, Austin, Texas) may be the platform used as a high throughput detection system suitable for adaptation directly to the clinical laboratory. For example, the Luminex 100 instrument is interfaced with a Gilson 215 multiprobe 96 or 384 well plate reader system. Since samples are injected into the Luminex 100 at 8-at-a-time groups, the analysis of the panel of analytes is a particularly convenient system for data processing. It is possible to simultaneously perform more than 100 assays on a single sample using the versatility of the Luminex bead technology.

The various microsphere functionalities for coupling proteins afford the methodology for covalent attachment of the immunoprobe to the bead (i.e. anti-human alpha-2-macroglobulin and anti-C 1INH captured by bead-linked avidin). A second antibody specific for kallikrein (or other activated plasma proteinase) will contain a reporter (fluorochrome) group. When the alpha-2-macroglobulin/kallikrein and C INH/kallikrein complexes are captured, the reporter antibody will provide an accurate quantitation of the level of kallikrein complexes in the sample. A specific spectral fingerprint may be used to segregate the beads for counting each of the individual components in the panel. This process may be developed for each of the 7-8 antigens in the panel and the combined assays will constitute the liver injury panel. The conjugation procedures for attachment of the antibodies to the microbeads and the coupling of the fluoroprobes to the antibodies have been well described chemically.

Some of the first serial complement activation data ever collected was obtained from a number of liver transplant recipients and patterns of complement activation during rejection episodes or proximal to the detection of either CMV and HCV infections⁴. It was however unexpected to see marked elevations in the levels of C3a and/or C4a in samples obtained from “stable” liver transplant recipients (n=19) whose routine clinical evaluation had showed no signs of disease or rejection. On average the C3a/C4a values were markedly elevated for the group with a rather wide variation between individuals. Even more remarkable was the fact that only 8 of the 19 continued to show extensive in vitro activation of C3 and C4 EDTA plasma demonstrating that an active enzyme exists that is not being controlled by the plasma proteinase inhibitors. We now suspect that this enzyme(s) is part of the lectin pathway of complement activation and these enzymes are known as mannan-binding associated serine proteinases (MASP- 1 and MASP-2). Therefore, the measurement of both in vivo and in vitro complement activation is important for proper interpretation of the results.

Armed with the in vivo and in vitro data on C3a and C4a levels in plasma, combined with evidence of MASP-1 activation, one can identify the pathway of complement that has been activated. Such data will provide insight into the injury processes that lead to the activation process. For example, if only C3a is generated in vivo and no in vitro activation occurred, one could conclude that only the alternative pathway was activated and that a form of infection by bacteria, fungi or parasite was a likely source. When both C3a and C4a are activated in vivo but not in vitro, and no MASP-½ activation is evident, then the classical complement pathway has been activated and immune complex disease or immune injury would be concluded to be the cause. Finally, if C3a and C4a were generated in vivo and in vitro and MASP-1 was activated, then the lectin complement pathway would have been activated and enzymatic tissue injury from neuraminidase could be the cause resulting in serious damage to the host's own cells. Thus, one can use the data to not only define the mechanism but also the extent of injury, and with serial analysis determine the progress or resolution of the process.

The following examples describe and illustrate the methods and compositions of the invention. These examples are intended to be merely illustrative of the present invention, and not limiting thereof in either scope or spirit. Unless indicated otherwise, all percentages and ratios are by weight. Those skilled in the art will readily understand that variations of the materials, conditions, and processes described in these examples can be used.

### EXAMPLE 1

### CHARACTERIZATION OF PLASMA PEPTIDASE ACTIVITY

This example demonstrates that plasma peptidase activity is elevated in many liver transplant patients, but this activity is not provided by the C4 converting enzyme.

Patients. Materials and Methods. Blood samples were obtained from healthy non-transplant donors (NTD) and stable orthotopic liver transplant recipients (LTR) under an approved Human Subjects protocol (no. 96-293). Each donor was asked to sign an informed consent form agreeing to be an unidentified voluntary donor. The LTR were free of clinical rejection according to standard evaluations performed at the time that these samples were collected. The liver transplants had been performed in these individuals from 1 month to nearly 4 years prior to the time of sample collection (Table 1).

Blood samples were drawn into 5 ml EDTA tubes (Venoject; Terumo Corp., Elkton, Md.). The plasma was collected immediately by centrifugation at 2,000×g for 15 min at 4° C. The plasma samples were either processed immediately for analysis or stored at −70° C. Frozen samples were thawed at 4° C. prior to analysis.

The chromogenic substrate D-Ile-Pro-Arg-pNA (S2288), trypsin, lima bean trypsin inhibitor (LBTI), soybean trypsin inhibitor (SBTI), aprotinin, benzamidine, bdellin, hirudin, human α-2-macroglobulin, human C1 esterase inhibitor (C1INH), and Sephacryl S-300 were purchased from Sigma Chemical (St. Louis, Mo.). D-Pro-Phe-Arg-pNA (S2302) was purchased from Chromogenix-Instrumentation Laboratory (Milan, Italy). Futhan (FUT175) was obtained from Banyu Pharmaceutical (Tokyo, Japan). C1s and C4 was obtained from Advanced Research Technologies (San Diego, Calif.). Plasmin, thrombin, plasma kallikrein, tissue kallikrein, and D-Phe-Phe-Arg-chloromethylketone (PPACK II) were obtained from Calbiochem-Novabiochem Corp (La Jolla, Calif.). β-Factor XIIa (β-FXIIa) was kindly provided by Dr. Mary J. Heeb, The Scripps Research Institute. A mAb against human prekallikrein/kallikrein (13G11) was obtained from QED Bioscience Inc. (San Diego, Calif.). A horseradish peroxidase-conjugated goat anti-mouse IgG and Opti-4CN™ substrate were obtained from Bio-Rad Labs (Hercules, Calif.). Goat anti-human α2-macroglobulin, goat anti-human C1INH, sheep anti-human antithrombin III (AT III), human plasma prekallikrein, and corn trypsin inhibitor (CTI) were purchased from Enzyme Research Labs. Inc (South Bend, Ind.). All other chemicals, buffer salts and reagents were analytical reagent grade.

The substrate AGLTR-pNA structure was based on the C-terminal sequence of the human C4a molecule and was synthesized by Peptides International, Inc. (Louisville, Ky.). Cleavage of the substrate between Arg and pNA by serine proteinases having trypsin-like specificity releases the yellow-colored pNA that is monitored at a visible wavelength of 405 nm.

The peptidase activity in plasma samples or of the purified enzymes were routinely determined at 37° C. in a 96-well plate. Each well contained 0.5 mM substrate in 100 μl of Tris-buffered saline (TBS)-EDTA buffer (50 mM Tris/150 mM NaCl/10 mM EDTA, pH 7.8) and the reaction was monitored using a microplate reader (Titertek Multiscan, Labsystems, Needham Heights, Mass.).

The C4a levels in EDTA plasma samples were determined using commercial human C4a Biotrak™ RIA (Amersham Life Science, Arlington Heights, Ill.). Equal volumes of plasma sample and the precipitating reagent were mixed and incubated at room temperature (rt) for 5 min. The mixture was then centrifuged at 2,500×g and 4° C. for 15 min. The supernatant from each sample tube was collected for analysis and aliquots were mixed with Biotrak assay buffer. The ¹²⁵I-labeled C4a and specific Ab solutions were added to the plasma supernatant and incubated at rt for 30 min. A 50 μl aliquot of goat anti-rabbit Ab was added and the mixture was incubated for an additional 30 min at rt. Two ml of saline was added and the tube was centrifuged at 2,000×g for 10 min at 4° C. The supernatant was decanted and the pellet was counted for 1 min in a Cobra Autogamma Model 5002 scintillation counter (Packard Instrument, Meriden, Conn.). Analysis of each sample was performed in duplicate and the data was analyzed using RiaSmart software supplied by Packard Instruments.

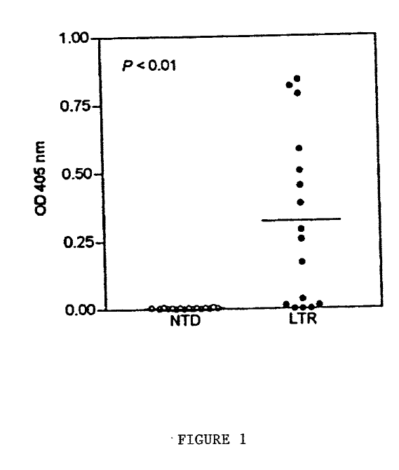

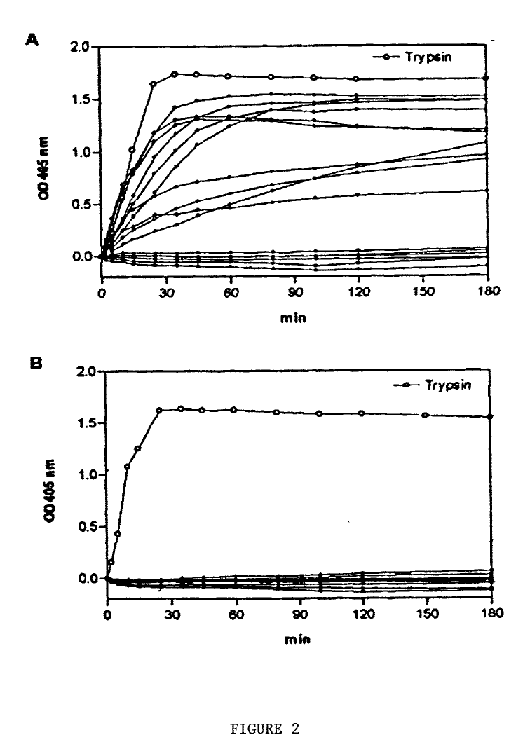

Characterization of plasma peptidase activity. Peptidase activities in EDTA plasma samples from NTD (n=16) and from LTR (n=16) were compared using the AGLTR-pNA substrate (FIG. 1). Activity was negligible in the NTD plasma samples (diluted 10-fold in TBS-EDTA buffer) after 15 min of incubation at 37° C. When the LTR EDTA plasma samples, also diluted 10-fold, were measured at the same early time point, a number of these samples (10/16, 63%) exhibited elevated peptidase activity. The mean peptidase activity measured in LTR plasma was significantly higher (P<0.01) than that in the NTD samples. The kinetic profiles of peptidase activity in LTR plasma diluted 10-fold in TBS-EDTA buffer show three distinct groups of samples (FIG. 2A). One group (n=6) has a high level of activity, and a second group (n=4) exhibits moderate activity, while the rest of the samples (n=6) show no activity at this dilution. No peptidase activity was detected in even undiluted NTD samples after 180 min of incubation at 37° C. (FIG. 2B).

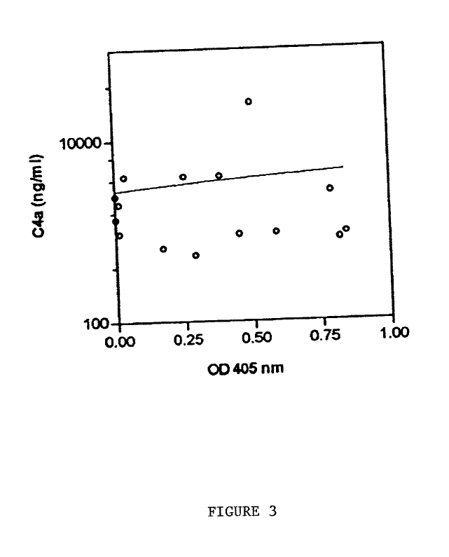

Correlation between plasma peptidase activity and C4a generation. If hydrolysis of the substrate AGLTR-pNA was a measure of the C4 converting enzyme in these LTR plasma, then the extent of in vitro complement activation, as measured by C4a generation , should correlate with the level of plasma peptidase activity. Since no correlation existed between the plasma C4a levels and the peptidase levels in these LTR plasmas (FIG. 3), it suggested that the plasma proteinase responsible for converting C4 was not the same enzyme that was being detected in the LTR plasma, even though we were using a substrate whose structure was based on the C4 cleavage site.

TABLE 1
Characteristics of the Liver Transplant Recipient Population

| | | | | | | Total post | |
|---|---|---|---|---|---|---|---|
| Recipient | Age* | Pre-transplant | Recurrence | Time post | | transplant | |
| No. | (yr) | diagnosis | of HCV/HBV | transplant^{‡} | | follow-up time | |
| 1 | 42 | Chronic HCV | Positive | 1 yr 1 | mo | 5 yr 2 | mo |
| 2 | 48 | Chronic HCV | Negative | 3 yr 3 | mo | 7 yr 4 | mo |
| 3 | 39 | Chronic HCV | Positive | 7 | mo | 4 yr 8 | mo |
| 4 | 49 | Chronic HCV | Positive | 4 | mo | 4 yr 5 | mo |
| 5 | 47 | Chronic HCV | Positive | 3 yr 9 | mo | 7 yr 10 | mo |
| 6 | 37 | Chronic HCV | Positive | 6 | mo | 4 yr 7 | mo |
| 7 | 39 | Chronic HCV | Positive | 5 | mo | 4 yr 7 | mo |
| 8 | 54 | Chronic HCV | Positive | 2 yr 0 | mo | 6 yr 1 | mo |
| 9 | 47 | Chronic active HCV | Negative | 3 yr 6 | mo | 7 yr 8 | mo |
| 10 | 47 | Chronic HCV and HBV | Negative | 2 | mo | 4 yr 4 | mo |
| 11 | 39 | Chronic HCV | Positive | 2 yr 3 | mo | 6 yr 4 | mo |
| | | Primary biliary cirrhosis | | | | | |
| 12 | 59 | Chronic HCV | Negative | 11 | mo | 5 yr 0 | mo |
| | | Autoimmune hepatitis | | | | | |
| 13 | 44 | Chronic HCV | Positive | 1 | mo | 4 yr 3 | mo |
| | | Systemic mastocytosis | | | | | |
| 14 | 59 | Chronic HCV | Positive | 2 | mo | 4 yr 3 | mo |
| | | Alcoholism | | | | | |
| 15 | 54 | Chronic HBV | Negative | 4 | mo | 4 yr 4 | mo |
| 16 | 48 | Autoimmune hepatitis | ND | 2 | mo | 4 yr 3 | mo |

*At time of transplant.
^{‡Length of time between transplant and when plasma was obtained.}

### EXAMPLE 2

### IDENTIFICATION OF THE PEPTIDASE ELEVATED IN CERTAIN LIVER TRANSPLANT PATIENTS AS KALLIKREIN OR A KALLIKREIN-LIKE PEPTIDASE

This example demonstrates that the peptides elevated in the plasma of many liver transplant patients is kallikrein or a kallikrein-like protein. levels of the kallikrein-like peptidase of the current invention are associated with liver disease.

Gel Filtration. Gel filtration experiments were performed at 4° C. and a flow rate of 10 ml per hour on a Sephacryl S-300 column (2.5×45 cm). The column was equilibrated with TBS-EDTA buffer and a 5 ml sample was applied to the column. Fractions of approximately 2.2 ml/tube were collected.

Immuno blot and immunodiffusion assays. Aliquots of the gel filtered plasma fractions were diluted in PBS and loaded (200 μl per well) onto a nitrocellulose membrane under vacuum. The membranes were blocked for 45 min with 5% nonfat dry milk in PBS. After washing with PBS containing 0.1% Tween-20, the blocked membranes were incubated with a mouse mAb to human prekallikrein/kallikrein (13G11) at 2 μg/ml in PBS containing 1% BSA for 90 min. After washing with PBS, the membranes were incubated for 45 min with a horseradish peroxidase-conjugated goat anti-mouse IgG at a dilution of 1/3000 in PBS containing 1% BSA. Finally, the membrane was washed with PBS and developed in Opti-4CN™ substrate. Each dot was quantitated with a computerized densitometer, Personal Densitometer SI (Molecular Dynamics, Sunnyvale, Calif.) using an image analysis software, ImageQuant (Molecular Dynamics).

A western blot analysis was performed using the mAb to human prekallikrein/kallikrein (13G 11). Human plasma prekallikrein was incubated with β-FXIIa (10:1 molar ratio) for 60 min at 37° C. Kallikrein-C1INH complexes and kallikrein-α-2-macroglobulin complexes were then formed by reacting the kallikrein with a twofold molar excess of C1INH and with a fivefold molar excess of α-2-macroglobulin, respectively, for 30 min at rt. SDS-PAGE was performed using the method of Laemmli²⁴. Samples were run on a 4% to 15% gradient SDS gel in nonreduced state and transferred to an Immobilon-P membrane (Millipore, Bedford, Mass.) by means of a semidry blotter (Hoefer Scientific Instruments, San Francisco, Calif.) following the manufacture's procedures. Immuno detection was conducted as described above.

Immunoprecipitation was performed using the Ouchterlony method²⁵. Agarose at a concentration of 1% in PBS containing 0.05% NaN₃ was poured onto plates. Twenty μl of polyclonal Ab to either human α-2-macroglobulin, C1INH, or AT III was placed in the center well of the gel plate. Ten μl of each fraction from the gel filtered plasma was placed in the surrounding wells. A positive result was based on the presence of a visible precipitation line between the Ab and the sample wells. The intensity of the precipitation lines were graded as ±, +, ++, or +++.

C4-cleaving assay. Each enzyme at 40 nM was incubated with a fixed concentration of C4 (1 μM) for 60 min at 37° C. in TBS buffer with EDTA, CaCl₂ or MgCl₂ added. An optimal pH of 7.4 was selected for the incubation of C1s and plasma kallikrein with C4. Following the incubation, SDS-PAGE was performed using the method of Laemmli²⁴. The disulfide bonds in C4 were reduced by incubating for 90 s at 95° C. in Laemmli's sample buffer supplemented with 5% 2-ME. The samples were applied to a 10% slab gel and after electrophoresis the protein was fixed and stained with Coomassie brilliant blue.

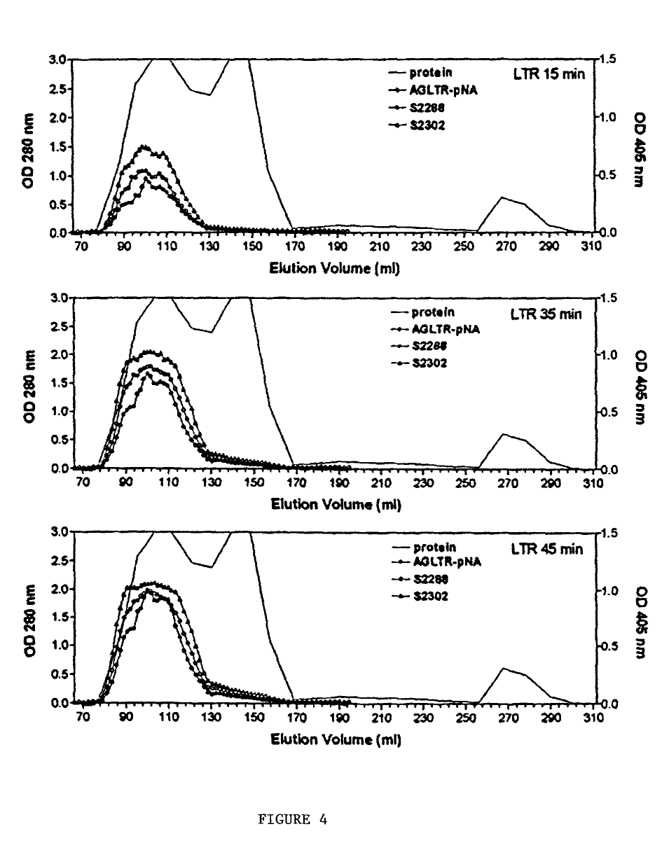

Gel filtration of plasma. One of the LTR samples containing high levels of peptidase activity was gel filtered and the profile of peptidase activity was determined using three different substrates (FIG. 4). The AGLTR-pNA peptidase activity in LTR plasma eluted over a broad range (80-125 ml). A similar profile was obtained when other of the high activity LTR samples were separated by the same gel filtration technique (data not shown). The size of the peptidase-containing material, based on known standards, was estimated to cover a broad range between 100-2,000 kD, indicating a degree of aggregation must exist. Although the rate of conversion measured using the commercial substrates S2288 or S2302 was somewhat greater than that using AGLTR-pNA, the activity profiles overlapped. The level of activity detected in the peak fractions of the gel filtered LTR plasma was relatively high after only 15 min of incubation at 37° C. Maximal peptidase activity was reached for the peak fractions after 35-45 min of incubation.

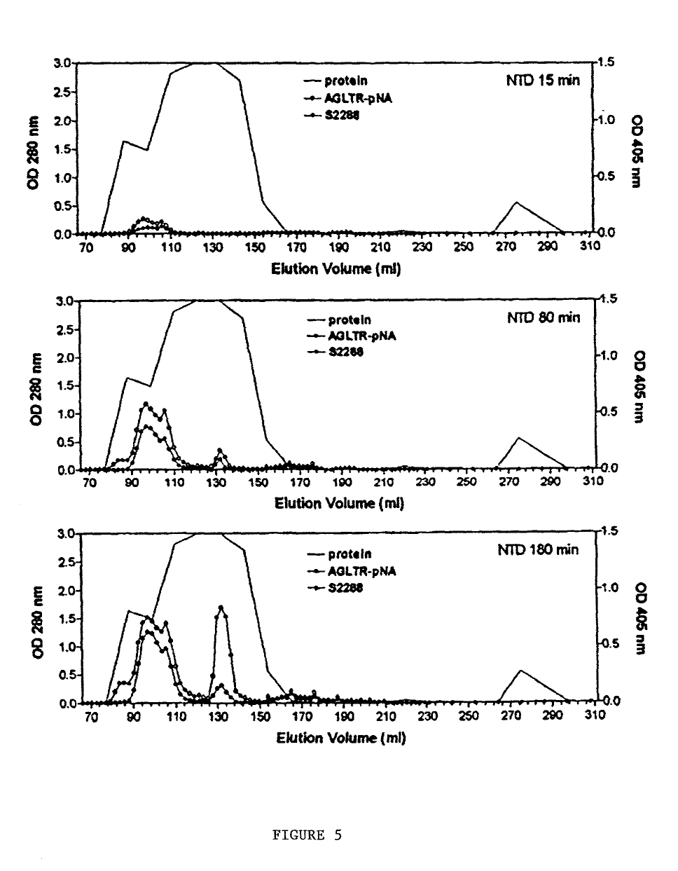

When the peptidase was measured in fractions of gel filtered NTD plasma, activity was barely detected after 15 min of incubation (FIG. 5). After long term incubation (80-180 min) activity was observed in a high mol wt region (90-110 ml, 200-1,000 kD), more narrow that in the LTR samples, as well as in a separate peak of activity at 130 ml (66 kD). Note that activity in the NTD plasma peak at 130 ml was much higher for the plasma thrombin substrate S2288 than for AGLTR-pNA. Therefore, not only is there much less activity detected in the NTD plasma, but the profiles of activity in LTR and NTD plasma are markedly different. We conclude that the majority of activity detected in the NTD plasma sample was generated in vitro.

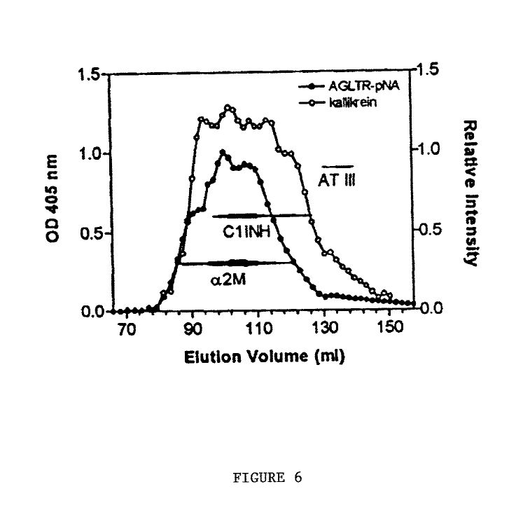

Plasma proteinase inhibitor profile. Immunological analysis of the peptidase-positive fractions of the LTR plasma samples by the Ouchterlony method²⁵ after gel filtration indicated that alpha-2-macroglobulin (α2M) coincides with the entire profile of peptidase activity, while C1INH only partially overlapped with the activity region, and AT III fell outside the region (FIG. 6). The profile of immunoreactive plasma kallikrein, as analyzed by the dot-blot technique, also overlapped with the peptidase activity. Both α2M and C1INH would be predicted to form proteinase-inhibitor complexes, but only the α2M-proteinase complexes retain peptidase activity.

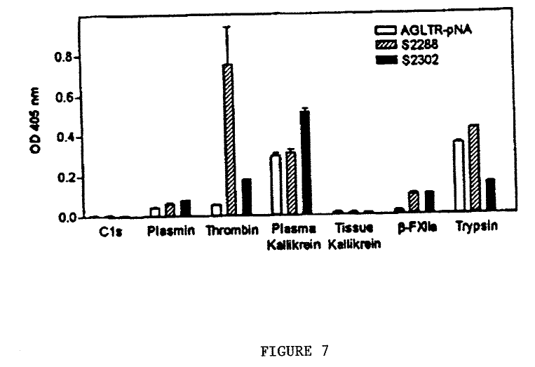

Activity of plasma proteins of synthetic substrates. AGLTR-pNA was cleaved efficiently by purified plasma kallikrein and trypsin, but poorly cleaved by thrombin and plasmin. Enzymes such as β-FXIIa, tissue kallikrein and C1s, the enzyme that the substrate was designed for, show almost no ability to cleave this substrate at these levels of 10 nM (FIG. 7). Substrates S2302 and S2288 were also cleaved by the purified enzymes at variable rates, with C1s, plasmin, tissue kallikrein and β-FXIIa being least active on these substrates. Thrombin and trypsin cleaved substrate S2288 more efficiently than S2302, while plasma kallikrein cleaved S2302 more rapidly than S2288. This was the same specificity that was observed for the LTR plasma peptidase.

Characterization of serine proteinase inhibitors on selected proteinase and liver transplant recipient plasma. Several common proteinase inhibitors have been evaluated for their ability to inhibit both the purified enzymes and the proteinase(s) in EDTA plasma from LTR (Table 2). Benzamidine and Futhan were very efficient inhibitors for all of the enzymes examined. LBTI inhibited the amidolytic activities of only plasmin and trypsin. The activity of plasma kallikrein was actually increased by adding LBTI. Both SBTI and aprotinin efficiently inhibited plasmin, plasma kallikrein, and trypsin, but not C1s. These inhibitors were only partially effective in inhibiting the plasma peptidase. All three of the LTR plasma samples that were examined exhibited a similar response pattern to these various inhibitors.

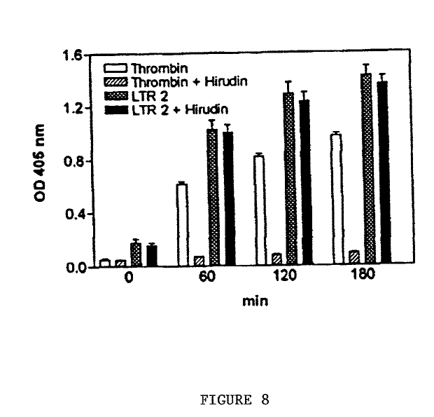

We examined several specific inhibitors of known plasma proteinases (Table 3). Many of the inhibitors in Table II had poor reactivity towards thrombin. However hirudin, a very effective thrombin inhibitor, failed to reduce the plasma peptidase activity indicating that thrombin was not involved (FIG. 8). It was important to eliminate thrombin as the proteinase detected here since thrombin activation has been correlated with cirrhotic liver patients infected with HCV²⁶. C1INH caused only partial inhibition of the plasma enzyme activity or purified plasma kallikrein, and only partially inhibited C1s at the concentration used here. Bdellin, a plasmin inhibitor, also failed to inhibit the LTR peptidase activity. The inhibitor PPACK II totally inhibited both purified plasma kallikrein and the LTR peptidase activity, but failed to inhibit C1s. This data strongly suggested that the LTR plasma enzyme was kallikrein and not C1s. The plasma peptidase activity could also be detected in citrated or heparin plasma, as well as in EDTA plasma (data not shown).

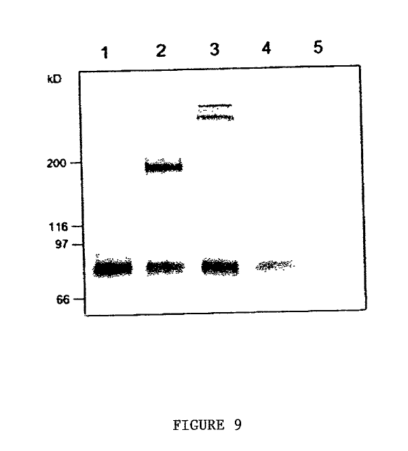

Immunologic detection of kallikrein-inhibitor complexes in liver transplant recipient plasma. It was previously reported that mAb 13G11 recognizes an epitope on the H chain of prekallikrein even when kallikrein is complexed with C1INH or with α2M^{27,28}. An immuno blot using purified kallikrein showed that the mAb readily detects plasma kallikrein (lane 1, a doublet at 86 and 88 kD), kallikrein-C1INH complexes (lane 2, 190 kD), and kallikrein-C1INH complexes, and kallikrein-α2M complexes (lane 3, multiple bands) (see FIG. 9). This result is consistent with the report by Kaufman et al²⁷. Note that there are aggregates of the kallikrein-α2M complexes visible in lane 3 which may help to explain the gel filtration patterns seen in FIG. 4. In LTR plasma (lane 5), the mAb detected prekallikrein/kallikrein, kallikrein-C1INH complexes, and kallikrein-α2M complexes while in NTD plasma (lane 4) only a prekallikrein/kallikrein doublet was visualized. In addition, the intensity of the prekallikrein/kallikrein in LTR plasma was significantly decreased compared to that in NTD.

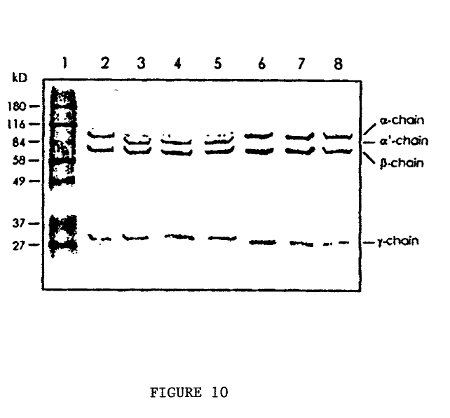

Attempt to cleave C4 by plasma kallikrein. Purified human C4 was incubated with either C Is or plasma kallikrein and cleavage of C4 was evaluated by SDS-PAGE using reduced condition (FIG. 10). As expected, control C4 without enzyme contained three chains (α-, β- and γ-) of 93, 75, and 32 kD respectively (lane 2). Treatment of C4 with C1s either with EDTA (lane 3), CaCl₂ (lane 4), or CaCl₂ and MgCl₂ (lane 5) resulted in cleavage of the C4 α-chain, producing the α′-chain. This result is consistent with previous reports of C4 conversion by C1s^{29,30}. However, plasma kallikrein failed to cleave C4 either with EDTA (lane 6), CaCl₂ (lane 7), or CaCl₂ and MgCl₂ (lane 8). Consequently, if plasma kallikrein is the peptidase detected in these LTR samples, as the data suggests, then a separate enzyme such as C1s or mannose-binding lectin-associated serine protease 2 (MASP-2) of the lectin complement activation pathway³¹ must be responsible for the in vitro cleavage of C4 that has been previously demonstrated in EDTA plasma⁴.

TABLE 2
Effects of General Serine Proteinase Inhibitors on
Selected Proteinases and LTR Plasmas

| Enzyme or | % Residual activity | | | | |
|---|---|---|---|---|---|
| Plasma | LBTI | SBTI | Aprotinin | Benzamidine | Futhan |
| C1s | 142.3 | 123.6 | 104.8 | 6.6 | 4.6 |
| Plasmin | 4.4 | 3.7 | 2.4 | 3.9 | −1.5 |
| Thrombin | 92.2 | 46.7 | 84.6 | 1.6 | 0.8 |
| Plasma Kallikrein | 284.2 | 2.2 | 3.5 | 2.0 | 1.1 |
| β-FXIIa | 68.7 | 109.1 | 106.0 | 10.9 | 3.8 |
| Trypsin | 4.8 | 5.3 | 4.8 | 5.8 | −0.5 |
| LTR 1 | 95.0 | 44.2 | 35.3 | 0.5 | −0.7 |
| LTR 2 | 95.2 | 61.9 | 40.2 | −0.3 | 0.2 |
| LTR 3 | 94.6 | 28.8 | 21.4 | 1.5 | −1.1 |

The enzymes C1s (100 nM), plasm in (100 nM), thrombin (100 nM), plasma kallikrein (20 nM), β-FXIIa (100 nM), trypsin (16 nM) or LTR EDTA plasma samples were incubated for 60 min at 4° C. with TBS-EDTA buffer containing either LBTI (1 mg/ml), SBTI (I mg/ml), aprotinin (0.075 mg/ml), benzamidine (50 mM), or Futhan (0.1 mg/ml), before mixing with the substrate. EDTA plasma samples from LTR were diluted 10 or 20-fold with TBS-EDTA. The chromogenic substrate AGLTR-pNA was used for all enzymes and LTR samples except C1s where the substrate S2288 was used. Based on the initial rate of increase in OD at 405 nm, the residual free enzyme level was calculated and expressed as a percentage of total activity without inhibitor added.

TABLE 3
Effects of Specific Serine Proteinase Inhibitors
on Selected Proteinases and LTR Plasmas

| | % Residual activity | | | | |
|---|---|---|---|---|---|
| Enzyme or Plasma | C1INH | Bdellin | Hirudin | PPACK II | CTI |
| C1s | 37.8 | ND | ND | 82.2 | ND |
| Plasmin | ND | 49.2 | ND | 9.2 | ND |
| Thrombin | ND | ND | 2.6 | 0.7 | ND |
| Plasma Kallikrein | 37.0 | ND | ND | 0.0 | ND |
| β-FXIIa | ND | ND | ND | 8.4 | 9.7 |
| LTR 1 | 81.4 | 99.1 | 100.4 | −0.2 | 96.4 |
| LTR 2 | 84.0 | 96.1 | 99.5 | 0.6 | 99.2 |
| LTR 3 | 41.0 | 103.4 | 104.1 | 2.7 | 104.5 |

Ten-fold diluted normal EDTA plasma was added to each enzyme. The enzymes C1s (100 nM), plasmin (100 nM), thrombin (100 nM), plasma kallikrein (20 nM), β-FXIIa (100 nM), trypsin (16 nM) or LTR EDTA plasma samples were incubated for 60 min at 4° C. with TBS-EDTA buffer containing either C1INH (40 μg/ml), bdellin (50 μg/ml), hirudin (50 U/ml), PPACK 11(10 μM), or CTI (100 μg/ml), before adding the substrate. LTR samples were 10 or 20-fold diluted with TBS-EDTA. The chromogenic substrate AGLTR-pNA was used for all enzymes and LTP samples except C1s where the substrate S2288 was used. Based on the initial rate of increase in OD at 405 nm, the residual free enzyme was calculated and expressed as a percentage of total activity determined without inhibitor added.

### EXAMPLE 3

### CORRELATIONS BETWEEN KALLIKREIN AND CLINICAL CONDITIONS

This example demonstrates that levels of the kallikrein-like peptidase of the current invention are associated with liver damage.

Determination of Clinical Outcome of Patient. Patients with clinical recurrent HCV and HBV infections were identified histologically by biopsy.

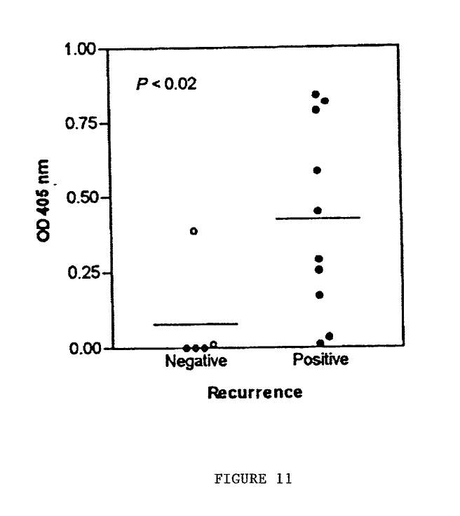

Correlations between kallikrein and clinical conditions. We investigated the relation between the peptidase activity and the clinical data (see Table 1) in LTR. There was no correlation between the peptidase activity and either rejection (5 of the 16 recipients, numbers 1, 4, 10, 11 and 15) or CMV infection (5 of the 16 recipients, numbers 2, 3, 4, 12 and 16) (see Table 4). All LTR that were examined, except recipient number 16 with autoimmune hepatitis, had HCV and/or hepatitis B virus (HBV) infection before liver transplantation and 10 of the 15 recipients were HCV recurrence positive. The peptidase activity in LTR with viral recurrence was significantly higher than in LTR without the recurrence (FIG. 11). Table IV summarizes these statistical correlates between complement activation and plasma kallikrein activity and the recurrence of HCV infections, CMV infections and rejection.

TABLE 4
Statistical Correlations between Plasma Kallikrein and
C4a Levels and Viral Infection and Rejection in LTR*

P values

| Condition | Kallikrein activity‡ | C4a level |
|---|---|---|
| HCV recurrence | 0.0164§ | 0.1573 |
| CMV infection | 0.2800 | 0.2945 |
| Rejection episodes | 0.1552 | 0.0195§ |

*The Mann-Whitney U-test was used.
‡The values for the peptidase activity shown in FIG. 1 were used.
§Statistically significant values.

### EXAMPLE 4

### FURTHER CONSIDERATIONS REGARDING PLASMA ENZYME LEVEL ELEVATION IN CERTAIN LIVER TRANSPLANT PATIENTS

Not to be limited by theory, this example discusses the results presented in FIGS. 1-3.

We previously reported significant elevation in the average levels of complement activation factors C3a and C4a in EDTA plasma from clinically stable liver transplant recipients⁴. There was also an unexpectedly high in vitro conversion of the complement factors C3 and C4 versus time in some of these LTR plasma samples. The incidence of markedly elevated C3a/C4a levels in LTR was 8 of 19 (42%), while the incidence in elevated peptidase activity in the same LTR plasmas appears to be even higher (62.5% or 10 of 16). Interestingly, there was no apparent correlation between these two processes in this group of transplant recipients who were clinically diagnosed as being free of rejection at the time that these samples were obtained. When EDTA plasma from the non-transplant control group of individuals were incubated in vitro, the level of plasma peptidase activity remained virtually undetectable.

Although low levels of activity were detected in NTD plasma following gel filtration, both the level and profile of activity differed significantly from that of the LTR plasma. It appears that low levels of peptidase activity was generated during the gel filtration process itself, since no activity was detected during incubation of the whole NTD plasma. This activation may simply have resulted from surface activation or the effect of dilution on the plasma³². Since the peptidase in the LTR plasma eluted over a wide range of mol wt and all appeared to be greater than 100 kD, it indicated that the enzyme was in complex with plasma proteinase inhibitors. In our assays, we used only small peptide substrates because the proteinase(s) in these complexes should be inactive toward protein substrates, as is known to be the case for α2M-proteinase complexes³³.

Unlike C1s and the MASP-1 and MASP-2 of the lectin pathway of complement activation, many plasma proteinases fail to cleave C3 or C4 efficiently, and some of the LTR plasma samples exhibited high levels of peptidase activity without generating C3a or C4a. This observation suggested that several enzymes were activated in the LTR plasma samples. In some cases, active C1s (or another C4 converting enzyme such as MASP-2 of the lectin pathway) was present while in other cases non-complement peptidases (i.e. kallikrein) had been activated. The proteinase inhibitor studies suggested that several proteinases could exist in LTR plasma, but that C1s, plasmin and thrombin appear to have been eliminated as candidates in these samples. It was important to identify the non-complement proteinase(s) in the LTR plasmas to help characterize the mechanisms underlying these activation processes. Knowing the enzyme(s) involved may eventually help us understand the nature of tissue injury responsible for activating complement and/or coagulation proteinases in liver transplants. Evidence that thrombin fragments F1+2, markers of thrombin activation³⁴, were elevated in patients with HCV infection with and without liver cirrhosis²⁶ suggested that thrombin may also be activated in the LTR. Thrombin would predictably be in complex with AT III and thus undetectable by our peptidase assay. We did not measure the F1+2 levels in the LTR samples and so we do not have evidence of thrombin being activated.

Our results from Western blot analysis clearly indicated that plasma kallikrein was activated and that it contributes most of the peptidase activity in LTR plasma based on our inhibitor studies. Furthermore, the evidence shows that the plasma kallikrein is complexed with various plasma inhibitors. This conclusion is consistent with the observation that plasma kallikrein is complexed primarily with C1INH and α2M when activated in normal plasma³⁵. The α2M-kallikrein complex has been measured in a number of diseases, including hereditary angioedema and sepsis, and found to be significantly elevated in a number of these cases²⁷. What is particularly interesting in these studies is that the enzyme-inhibitor complexes are observed in only a select population of the LTR samples indicating that the activation process is dynamic and is signaling a specific pathologic event in stable LTR.

A recent study suggests that a factor XII-independent pathway of prekallikrein activation exists on the surface of cultured human endothelial cells and that the activating enzyme may be a cellular thiol enzyme³⁶. This observation may explain how and why the peptidase activity is elevated only in a select group of LTR plasma from recipients with recurrent HCV infection (Table 4). If prekallikrein is activated on endothelium after the cells are injured, causing release of the thiol enzyme(s)³⁶, then this peptidase activity may be used to indicate ongoing endothelial injury. Alternatively, the activating cells may be the HCV-infected hepatocytes themselves that can either generate kallikrein by contact activation on the injured cell surface or by elaborating the HCV trypsin-like proteinase NS3^{37;38}. It has even been suggested that the NS3 protease could deplete plasma of inhibitors such as C1INH and α2-antiplasmin and thereby altering normal regulatory mechanisms of the complement and coagulation cascades³⁸.

Consequently, our data suggests that either plasma peptidase activity or direct measurement of the α2M-kallikrein complexes in LTR plasma provide a means to monitor pathologic events leading to hepatic or endothelial injury. Kallikrein activation appears to be a separate event from the rejection or immune responses leading to complement activation. Although Scholz et al.²² detected contact activation of kallikrein immediately after reperfusion of the transplanted liver, our data suggests that similar episodes can occur much later in the life of the graft and are independent of revascularization.

Since elevated peptidase activity was detected only in some of the recipients, all of which were clinically diagnosed as stable and non-rejecting at the time the sample was obtained, it was important to examine this population prospectively for events that might correlate with elevated enzyme levels. A significant (P<0.02) correlation was found between recurrence of HCV infection in the majority of these recipients (10 of 15) and elevated peptidase activity. We did not find a correlation between rejection episodes (observed in only 5 of 16 recipients) and peptidase activity, however rejection did correlate significantly (P<0.02) with C4a generation¹. Since no evidence of C1s activity was observed (i.e. classical pathway activation), this result suggests that the C4 may be converted by the MASP-2 enzyme of the lectin pathway³¹.

A number of questions have been raised by these studies, such as how many proteinases are actually involved and why are hemostatic system and coagulation enzymes activated in some cases while complement is activated in others. We also need to know if the elevation in kallikrein activity might have diagnostic value for these organ recipients. Does the presence of kallikrein and other peptidases signal early stages of viral injury to hepatocytes or to the vascular endothelial surface and what pathophysiologic events lead to such vascular injury in this organ? Our results suggest that serial measurements of the peptidase activity and/or levels of α2M-kallikrein complexes in LTR may be used to correlate with clinical sequelae. Both the peptidase and complement activation assays promise to be useful in discerning stages of progressive disease in liver allografts. This is the first evidence that kallikrein activation is significantly correlated with HCV infection in liver transplants.

### EXAMPLE 5

### PANEL ASSAY FOR DETECTING LIVER DISEASE

This example provides a prophetic example of a panel assay for detecting, monitoring, and diagnosing liver disease. The purpose of the experiments described in this experiment are to obtain and evaluate the specific immunoreagents for the 7-8 components of the panel assay.

Coupling experiments will be performed on microbeads and antibodies. Seven to eight assays will be adapted to the Luminex instrument. Activated plasma samples and purified components in the assay system will be tested for reproducibility and accuracy. Analyte sensitivity will be determined and used to establish detection limits for each analyte using standard statistical analysis. Calibrators will be developed for establishing assay accuracy and controls for assay reproducibility. Control and disease levels will be established for each of the component assays.

Experimental Protocol. A panel of assays using the Luminex microbead technology will be developed for analysis of plasma samples. Seven to eight protein factors have been selected based on existing data that shows a correlation between the levels of these factors and a known disease/injury process in the liver. The factors, also referred to herein as the liver damage panel, include complement components C3a, C4a, C1s and MASP-1, plasma prekallikrein and kallikrein/inhibitor (e.g. alpha-2-macroglobulin and C1 inhibitor) complexes, as well as serum amyloid protein (SAP) and/or C-reactive protein (CRP) representing acute phase proteins.

Development of the panel requires an optimization of the accuracy and reproducibility of the individual assays and a demonstration that the panel data is representative and duplicative of the individual assay results. The conjugation procedures for attachment of the antibodies to the microbeads and the coupling of the fluoroprobes to the antibodies have been well described chemically, however this process must be demonstrated using each of the protein factors selected for the panel.

The antibodies and reference factors will be obtained from either commercial sources or from personal stores. Antibodies will be characterized for desired binding affinities and specificity. Coupling of the antibodies to the microbeads will be efficient and at a density that is appropriate to meet the binding/uptake requirement for each factor. Attachment of the fluoroprobes to the antibodies will be uniform and sufficient in number to permit adequate sensitivity. The panel of assays will be evaluated and shown to be reliable, reproducible, and accurate compared to independent assay techniques. The stability and storage characteristics of the coupled reagents will meet standards that permit reproducibility of assay results over a span of at least 60 days.

Potential application of the panel assays will be demonstrated using both control (i.e. normal) and activated plasma. Both complement and pre-kallikrein will be activated in the test plasma samples to simulate the data that has been obtained from liver transplant recipients or liver disease plasmas.

Design and development of the individual assays in the panel. A complete set of reagents for characterization will be obtained as follows:

1. Purified components such as C3a, C4a, C1s, Masp-1, prekallikrein, kallikrein, SAP and CRP will be obtained from sources such as Advanced Research Technologies, San Diego, Calif. (a complement source) or Enzyme Research (South Bend, Ind. (an enzyme source) or BD/Pharmingen (an antibody source).

2. All components will be characterized for purity and authenticity using mass spectroscopy and gel electrophoresis.

3. All antibodies will be tested in competition assays to determine their value in the sandwich procedure.

Immunoglobulins will be attached to the beads via either a carboxyl group or to the Lumavidin. The level of antigen captured by the reagent beads will be tested.

Antibody will be labeled with Bodipy FL-CASE@ from Molecular Probes, Inc., Eugene, Oreg. Intensity of the green fluorescence on the antibody will be measured.

The capture microspheres will be used to titrate the component antigen. Uptake curves for the component will be generated and statistical methods will be used to determine sensitivity of the assay.

This process will be repeated for each component of the assay panel.

The assays will then be adapted to the multiplex panel for automatic data collection. The various capture microbeads will be mixed with the various purified components and the reporter antibodies. The assay values, reproducibility, and accuracy of the output will be determined.

The prototype panel assay will then be analyzed using plasma samples. Various capture microbeads for the 7-8 components will be mixed with the plasma samples (activated and non-activated) and add the reporter antibodies. The assay values, reproducibility, and accuracy of the data output will be determined.

As many as 50 normal human donors to provide 5-10 ml of blood collected in EDTA. The distribution of subjects should be approximately 50% male and 50% female between the ages of 18-55 with no preference to race. The expected distribution from the general population will be approximately 15% African-American, 20% Asian, 25% Hispanic and 40% Caucasian based on the local population.

The 5-10 ml blood sample will be drawn from the antecubital vein or another arm vein, if necessary. Blood will be drawn at the San Diego Blood Center and subjects will be recruited from the population at large. A standard consent form approved by the Blood Center will be used. The protocols are already developed at HemoSaga for such studies (Assurance of Compliance No. FWA00000150).

The potential risks are minimal and consist primarily of a slight temporary bruise appearing around the venipuncture site. The SD Blood Center maintains a trained staff of certified phebotomists.

Throughout this application, various patents, publications, books, and nucleic acid and amino acid sequences have been cited. The entireties of each of these patents, publications, books, and sequences are hereby incorporated by reference into this application.

### REFERENCES

1. Pfeifer P H, Kawahara M, Hugli T E. Possible mechanism for in vitro complement (C) activation in blood and plasma samples: Futhan/EDTA controls in vitro complement activation. Clinical Chemistry. 1999; 45: 1190-1199.

2. Ikari N, Sakai Y, Hitomi Y, Fujii S. New synthetic inhibitor to the alternative complement pathway. Immunology. 1983; 49:685-691.

3. Hitomi Y, Fujii S. Inhibition of various immunological reactions in vivo by a new synthetic complement inhibitor. Int Arch Allergy Appl Immunol. 1982; 69:262-267.

4. Pfeifer P H, Brems J J, Brunson M, Hugli T E. Plasma C3a and C4a levels in liver transplant recipients: A longitudinal study. Immunopharmacology. 2000; 46:163-174.

5. Moon K E, Gorski J P, Hugli T E. Complete primary structure of human C4a anaphylatoxin. J Biol Chem. 1981; 256:8685-8692.

6. Petry F, Loos M. Bacteria and complement. Volanakis J E, Frank M M, eds. In: The human complement system in health and disease, New York, Marcel Dekker, Inc., 1998: 375-391.

7. Cooper N R. Complement and viruses. Volanakis J E, Frank M M, eds. In: The human complement system in health and disease, New York, Marcel Dekker, Inc., 1998: 393-407.

8. Schenkein H A. The role of complement in periodontal diseases. Crit Rev Oral Biol Med. 1991; 2:65-81..

9. Franco A G, Schur P H. Hypocomplementemia in rheumatoid arthritis. Arthritis Rheum. 1971; 14:231-238.

10. Buyon J P, Tamerius J, Ordorica S, Young B, Abramson S B. Activation of the alternative complement pathway accompanies disease flares in systemic lupus erythematosus during pregnancy. Arthritis Rheum. 1992; 35:55-61.

11. Moxley G, Ruddy S. Elevated plasma C3 anaphylatoxin levels in rheumatoid arthritis patients. Arthritis Rheum. 1987; 30:1097-1104.

12. Atkinson J P. Complement activation and complement receptors in systemic lupus erythematosus. Semin Immunol Pathol. 1986; 9:179-194.

13. Townes A S, Stewart C R. Estimations of the second component and third component complex of complement in the serum of patients with systemic lupus erythematosus. Bull.Hopkins Hosp. 1965; 117:348-362.

14. Hack C E, Nuijens J H, Felt-Bersma R J F, et al. Elevated plasma levels of the anaphylatoxins C3a and C4a are associated with fatal outcome in sepsis. Am J Med. 1989; 86:20-26.

15. Heideman M, Hugli T E. Anaphylatoxin generation in multisystem organ failure. J Trauma 1984; 24:1038-1043.

16. Chenoweth D E, Cooper S W, Hugli T E, Stewart R W, Blackstone E H, Kirklin J W. Complement activation during cardiopulmonary bypass: evidence for generation of C3a and C5a anaphylatoxins. N Engl J Med. 1981; 304:497-503.

17. Craddock P R, Fehr J, Brigham K L, Kronenberg R S, Jacob H S. Complement and leukocyte-mediated pulmonary dysfunction in hemodialysis. N Engl J Med. 1977; 296:769-774.

18. Nusbacher J, Rosenfeld S I, MacPherson J L, Thiem P A, Leddy J P. Nylon Fiber leukapheresis: associated complement component changes and granulocytopenia. Blood. 1978; 51:359-365.

19. Hammerschmidt D E, Craddock P R, McCullough J, Kronenberg R S, Dalmasso A P, Jacob H S. Complement activation and pulmonary leukostasis during nylon fiber filtration leukapheresis. Blood. 1978; 51:721-730.

20. Kirschfink M, Wienert T, Rother K, Pomer S. Complement activation in renal allograft recipients. Transplantation Proceedings 1992; 24:2556-2557.

21. Ronholm E, Tomasdottir H, Runeborg J, et al. Complement system activation during orthotopic liver transplantation in man. Transplantation. 1994; 57:1594-1597.

22. Scholz T, Backman L, Mathisen O, et al. Activation of the plasma contact system and hemodynamic changes after graft revascularization in liver transplantation. Transplantation 1995; 60:36-40.

23. Hayashi J, Hugli T E. Kallikrein-like activity associated with ∀-2-macroglobuilin (∀-2-M) in plasma from liver transplant recipients [abstract]. Protein Science 2000; 9:462-T.

24. Laemmli U K. Cleavage of structural proteins during the assembly of the head of bacteriophage T4. Nature. 1970; 227:680-685.

25. Ouchterlony O, Nilsson L -A. Immunodiffusion and immunoelectrophoesis. Weir D M, Herzenberg L A, Blackwell C, eds. In: Handbook of Experimental Immunology, Oxford, Blackwell, 1986; 32.1-32.5.

26. Violi F, Ferro D, Basili S, et al. Increased rate of thrombin generation in hepatitis C virus cirrhotic patients. Relationship to venous thrombosis. J Invest Med. 1995; 43:550-554.

27. Kaufman N, Page J D, Pixley R A, Schein R, Schmaier A H, Colman R W. ∀2-macroglobulin-kallikrein complexes detect contact system activation in hereditary angioedema and human sepsis. Blood. 1991; 77:2660-2667.

28. Veloso D, Silver L, Hahn S, Colman R. A monoclonal anit-human plasma prekallikrein anitbody that inhibits activation of prekillkrein by factor XIIa on a surface. Blood. 1987; 70:1053-1062.

29. Budzko D B, Bokisch V A, Muller-Eberhard H J. A fragment of the third component of human complement with anaphylatoxin activity. Biochemistry. 1971; 10:1166-1172.

30. Patrick R A, Taubman S B, Lepow I H. Cleavage of the fourth component of human complement (C4) by activated C1s. Immunochemistry. 1970; 7:217-225.

31. Reid K B M. C1q and mannose-binding lectin. Volanakis J, Frank M, eds. In: The human complement system in health and disease, New York, Marcel Dekker, Inc., 1998; 33-48.

32. Renaux J- L, Thomas M, Crost T, Loughraieb N, Vantard G. Activation of the kallikrein-kinin system in hemodialysis: Role of membrane electronegativity, blood dilution, and pH. Kidney Int. 1999; 55:1097-1103.

33. Harpel P C, Lewin M F. Distribution of plasma kallikrein between C1 inactivator and ∀2-macroglobulin in plasma utilizing a new assay for □2-macroglobulin-kallikrein complexes. J Biol Chem 1985; 260:4257-4263.

34. Teitel J M, Bauer K A, Lau H K, Rosenberg R D. Studies of the prothrombin activation pathway utilizing radioimmunoassays for the F1/F1+2 fragment and thrombin-antithrombin complex. Blood. 1982; 59:1086-1097.

35. Schapira M, Scott C F, Colman R W. Contribution of plasma protease inhibitors to the inactivation of kallikrein in plasma. J Clin Invest 1982; 69:462-468.

36. Motta G, Rojkjaer R, Hasan A A K, Cines D B, Schmaier A H. High molecular weight kininogen regulates prekallikrein assembly and activation on endothelial cells: A novel mechanism for contact activation. Blood. 1998; 91:516-528.

37. Love R A, Parge H E, Wickersham J A, et al. The crystal structure of hepatitis C virus NS3 proteinase reveals a trypsin-like fold and a structural zinc binding site. Cell. 1996; 87:331-342.

38. Drouet C, Bouillet L, Csoopaki F, Colomb M G. Hepatitis C virus NS3 serine protease interacts with the serpin C1 inhibitor. FEBS Lett. 1999; 458:415-418.

39. Carson, R. T. and D. A. A. Vignali Simultaneous quantitation of 15 cytokines using a multiplexed flow cytometric assay. J. of Immunological Methods 227: 41-52 (1999).

40. J. E. Volanakis Overview of the Complement System. in The Human Complement System in Health and Disease (Volanakis, J. E. and Frank, M. M., editors) Marcel Dekker, Inc. New York, N. Y.

41. J. Hayashi, D. R. Salomon, and T. E. Hugli. Elevated kallikrein activity in plasma from stable liver transplant recipients. Arterioscler. Thromb. .Vasc. Biol., in press.

42. E. Rodriguez-Iñigo, J. Bartolomé, S. de Lucas, F. Manzarbeitia, M. Pardo, C. Arocena, J. Gosálvez, H. Oliva and V. Carreño. Histological Damage in Chronic Hepatitis C is Not Related to the Extent of Infection in the Liver. Am. Journ. Pathology V. 154, 6:1877-1881.

43. K. B. M. Reid C1q and Mannose-Binding Lectin. In The Human Complement System in Health and Disease (Volanakis, J. E. and Frank, M. M., editors) Marcel Dekker, Inc. New York, N.Y.

44. T. E. Hugli, and R. B. Stoughton, U.S. Ser. No. 09/245,829, “Methods for Assessing Complement Activation,” filed Feb. 5, 1999.

What is claimed is:

1. A method for detecting liver damage in a subject, said method comprising contacting a sample from the subject with a plasma kallikrein-like peptidase detection reagent, wherein the kallikrein-like peptidase detection reagent detects the kallikrein-like peptidase, and wherein interaction of the kallikrein-like peptidase with the kallikrein-like peptidase detection reagent is indicative of liver damage in the subject.

2. The method of claim 1, wherein the kallikrein-like peptidase is plasma kallikrein.

3. The method of claim 1, wherein the kallikrein-like peptidase detection reagent is a substrate cleaved by kallikrein.

4. The method of claim 1, wherein the kallikrein-like peptidase detection reagent is a kallikrein binding reagent.

5. The method of claim 2, further comprising contacting the sample with an alpha-2-macroglobulin detection reagent.

6. The method of claim 5, wherein kallikrein detected by the method is bound to alpha-2-macroglobulin.

7. The method of claim 1, wherein first sample is a blood or a plasma sample.

8. The method of claim 1, wherein the subject is a mammal.

9. The method of claim 1, wherein the subject is a stable liver transplant patient.

10. The method of claim 1, wherein the subject has a hepatitis infection.

11. The method of claim 1, wherein the subject has an HCV infection.

12. The method of claim 1, wherein the subject has a recurrent HCV infection.

13. The method of claim 1, wherein the subject has an HBV infection.

14. The method of claim 1, wherein the kallikrein-like peptidase detection reagent comprises a peptide.

15. The method of claim 1, further comprising detecting C3a, C4a, or a combination thereof.

16. The method of claim 1, wherein the subject has a liver disease selected from liver abscess, liver cancer, either primary or metastatic, cirrhosis, such as cirrhosis caused by the alcohol consumption or primary biliary cirrhosis, amebic liver abscess, autoimmune hepatitis, biliary atresia, coccidioidomycosis disseminated, delta agent (hepatitis d), hemochromatosis, hepatitis a, hepatitis b, hepatitis c, or other hepatitis virus, hepatocellular carcinoma, pyogenic liver abscess, Reye's syndrome, sclerosing cholangitis, Wilson's disease, drug induced hepatotoxicity, or fulminant or acute liver failure.

17. The method of claim 15, further comprising detecting interaction of the C4a detection reagent with C4a, wherein an elevated level of C4a indicates autoimmune liver damage.

18. The method of claim 2, wherein the method further comprises:
dividing the sample into a first portion and a second portion before contacting the sample with the substrate;
adding a kallikrein protease inhibitor to the first portion of the sample before contacting the sample with the substrate; and
comparing substrate cleavage in the first portion with substrate cleavage in the second portion, wherein a difference in substrate cleavage is indicative of liver damage.

19. The method of claim 18, wherein the kallikrein protease inhibitor is PPACK II.

20. The method of claim 1, further comprising contacting the sample with a prekallikrein detection reagent and detecting interaction of the prekallikrein with the prekallikrein detection reagent, wherein a difference in the level of the prekallikrein is indicative of liver damage in the subject.

21. The method of claim 4, wherein the kallikrein binding reagent comprises an anti-kallikrein antibody or an active site reactive reagent.

22. A kit comprising a first container containing a kallikrein-like peptidase detection reagent.

23. A kit comprising a first container containing a kallikrein-like peptidase detection reagent and a second container containing an alpha-2-macroglobulin detection reagent.

24. A kit comprising a first container containing a kallikrein-like peptidase detection reagent and a second container containing a C4a detection reagent or a C3a detection reagent, or a combination thereof.

25. A method for monitoring the progression of liver damage in a subject comprising:
contacting a first sample and a second sample with a kallikrein-like peptidase detection reagent, wherein the first sample is from a first timepoint and the second sample is from a second timepoint, and wherein the kallikrein-like peptidase detection reagent detects kallikrein-like peptidase; and
comparing the level of kallikrein-like peptidase in the first sample and the second sample, wherein a change in the relative quantity of the kallikrein-like peptidase is indicative of a change in liver damage state.

26. The method of claim 25, wherein the kallikrein-like peptidase is kallikrein.

27. The method of claim 26, further comprising contacting the sample with a plasma peptidase or protease inhibitor detection reagent.

28. The method of claim 27, wherein the inhibitor is alpha-2-macroglobulin, C1 inhibitor or alpha-I antitrypsin inhibitor or antithrombin.

29. The method of claim 28, wherein kallikrein measured by the method is bound to alpha-2-macroglobulin.

30. The method of claim 25, wherein the first sample and the second sample are a plasma or a blood sample.

31. The method of claim 25, wherein the subject is a mammal.

32. The method of claim 25, wherein the subject is a human.

33. The method of claim 25, wherein the subject is a stable liver transplant patient.

34. The method of claim 26, wherein the subject is a stable liver transplant patient.

35. The method of claim 25, wherein the subject has a hepatitis infection.

36. The method of claim 35, wherein the subject has an HCV infection.

37. The method of claim 35, wherein the subject has a recurrent HCV infection.

38. The method of claim 35, wherein the subject has an HBV infection.

39. The method of claim 25, further comprising contacting the first sample and the second sample with a C3a and a C4a detection reagent.

40. The method of claim 39, further comprising determining a relative quantity of C3a and C4a by detecting the amount of interaction of the C3a and C4a detection reagent with C3a and C4a, respectively.

41. A method for detecting kallikrein in a sample, said method comprising contacting the sample with an isolated tri, tetra or penta peptide containing an arginine residue at the P1 position, wherein the peptide comprises a peptidase substrate.

42. The method of claim 41, wherein the peptide further comprises a detectable moiety.

43. The method of claim 42, wherein the detectable moiety is an enzyme, radioisotope, fluorescent compound, colloidal metal, a chromogenic moiety, chemiluminescent compound, phosphorescent compound, or bioluminescent compound.

44. The method of claim 43, wherein the chromogenic moiety is pNA.

45. The method of claim 41, wherein the sample is a blood-based sample.

46. The method of claim 41, wherein the blood-based sample is a plasma sample.

47. A method for determining toxicity of a therapeutic agent in a subject comprising contacting a sample from the subject with a kallikrein-like peptidase detection reagent and determining a kallikrein-like peptidase level in the sample, wherein an elevated kallikrein-like peptidase level indicates toxicity of the therapeutic agent.

48. The method of claim 47, wherein the elevated kallikrein-like peptidase level is determined by comparing the kallikrein-like peptidase level of the sample with a kallikrein-like peptidase level of a control not containing kallikrein-like peptidase.

49. The method of claim 47, wherein the kallikrein-like peptidase is kallikrein.

50. The method of claim 47, wherein the kallikrein-like peptidase detection reagent is a substrate cleaved by kallikrein.

51. The method of claim 47, wherein the kallikrein-like peptidase detection reagent is a kallikrein binding reagent.

52. The method of claim 47, wherein the kallikrein binding reagent comprises an anti-kallikrein antibody.

53. The method of claim 47, further comprising contacting the sample with an alpha-2-macroglobulin detection reagent.

54. The method of claim 53, wherein kallikrein is bound to alpha-2-macroglobulin, C1 inhibitor, antithrombin or alpha 1 antitrypsin inhibitor.

55. The method of claim 47, wherein the first sample is a blood or plasma sample.

56. The method of claim 47, wherein the subject is a mammal.

57. A method for detecting liver damage in a subject, said method comprising contacting a sample from the subject with a series of detection reagents that are specific for each member of a liver damage panel comprising kallikrein, wherein an elevated level of one or more members of the liver damage panel is indicative of liver damage in the subject.

58. The method of claim 57, wherein the liver damage panel comprises complement components.

59. The method of claim 58, wherein the liver damage panel further comprises a mannan binding serine protease, C1 esterase, serum amyloid protein, and C-reactive protein.

60. The method of claim 59, wherein the complement components include C3a and C4a.

61. The method of claim 60 wherein the complement components further include activated mannan binding serine protease-1.

62. An in vitro method for screening a therapeutic agent for toxicity, said method comprising:
incubating a liver cell cultured in cell culture medium with the therapeutic compound to obtain sample cell culture medium;
contacting the sample cell culture medium with a kallikrein-like peptidase detection reagent, wherein interaction of the kallikrein-like peptidase with the kallikrein-like peptidase detection agent is indicative of therapeutic agent toxicity.

63. The method of claim 62, wherein the kallikrein-like peptidase is kallikrein.

64. The method of claim 65, wherein the kallikrein-like peptidase detection reagent is a substrate cleaved by kallikrein.

65. The method of claim 65, wherein the kallikrein-like peptidase detection reagent is a kallikrein binding reagent.

66. The method of claim 65, wherein the kallikrein binding reagent comprises an anti-kallikrein antibody.

67. The method of claim 63, further comprising contacting the sample with an alpha-2-macroglobulin, C1 inhibitor, antithrombin or alpha-1 antitrypsin detection reagent.

68. The method of claim 67, wherein kallikrein detected by the method is bound to alpha-2-macroglobulin, C1 inhibitor, antithrombin or alpha-1 antitrypsin.

69. The method of claim 2, further comprising contacting the sample with a C1 inhibitor detection reagent.

70. The method of claim 2, further comprising contacting the sample with a alpha 1 antitrypsin detection reagent.

71. The method of claim 2, further comprising contacting the sample with a antithrombin detection reagent.

72. The method of claim 42, wherein the detectable moiety is an antibody that detects the action of kallikrein on a peptide substrate.
